(19) United States
(12) Patent Application Publication
(10) Pub. No.: US 2026/0125409 A1
(43) Pub. Date: May 7, 2026

(54) METHOD OF ENHANCING THE BIOAVAILABILITY OF BIOACTIVE CHEMICAL COMPOUNDS AND THERAPEUTIC DRUGS, NOVEL BIOAVAILABLE COMPOUNDS, AND METHODS OF USE

(71) Applicant: GEROSYNTH LABORATORIES, INC., Bloomington, MN (US)

(72) Inventor: ALISHA S. O'DELL, Lakeville, MN (US)

(73) Assignee: GEROSYNTH LABORATORIES, INC., Bloomington, MN (US)

(21) Appl. No.: 19/378,852

(22) Filed: Nov. 4, 2025

Related U.S. Application Data
(60) Provisional application No. 63/716,799, filed on Nov. 6, 2024.
(60) Provisional application No. 63/717,272, filed on Nov. 6, 2024.

(51) Int. Cl. C07H 5/06; A61K 31/7056

(52) U.S. Cl. CPC C07H 5/06; A61K 31/7056

(57) ABSTRACT

Methods for chemically modifying bioactive triterpene compounds to increase the bioavailability thereof. The resulting triterpene chemical compounds, having enhanced bioavailability, are useful in pharmaceutical compositions, alone, in combination with, or complexed with therapeutic drugs for the treatment of diseases such as cancers.

### INCORPORATION BY REFERENCE

Applicant's pending PCT/US2024/027210, filed May 1, 2024, is incorporated by reference herein in its entirety.

### PRIORITY

The present application claims the full benefit and priority of U.S. provisional patent application Nos. 63/716,799 and 63/717,272, both filed on Nov. 6, 2024. They are likewise fully incorporated by reference.

### FIELD

The present invention relates generally to methods for producing, synthesizing or chemically modifying bioactive compounds to increase the bioavailability thereof. The resulting chemical compounds, having enhanced bioavailability, are useful in pharmaceutical compositions for the treatment of diseases such as cancers.

### BACKGROUND OF THE INVENTION

Here follows a preliminary discussion of the invention, which is not necessarily prior art, and should not necessarily be so construed.

Cancer is a leading cause of death worldwide. Additionally, an alarming rate of new cancer diagnosis in people at increasingly younger ages continues to threaten public health and quality of life. Current treatments for the human population can be effective, but there is still a significant failure rate with conventional treatments, many anti-cancer drugs currently available are not target specific and produce several side effects and complications, and the threat to long term health and quality of life remains.

Dermatologic disease and various equine cancers are growing at an unprecedented rate in various species. It is expected that there will be a 75%-90% increase in equine cancer diagnosis annually by 2030. There are no long-term treatment options for equines for advanced-stage cancer.

Therefore, there is a critical need for novel effective and less toxic therapeutic approaches to cancer treatment for both humans and animals, including equine animals.

The therapeutic index for a pharmaceutical drug is the amount of drug a patient requires in order to generate the desired therapeutic effect without causing a toxic effect. Drug toxicity occurs when the bioaccumulation threshold is exceeded, and the amount of drug necessary for the therapeutic effect becomes too much for the body to metabolize. When the rate of drug absorption needed for therapeutic effect exceeds the rate of drug metabolism, the drug accumulates in the body and causes toxic effects. Drugs with low bioavailability require higher doses, making it more likely that the drug will accumulate in the body and cause toxicity. By increasing the bioavailability of a therapeutic drug, a lower dose can be administered thereby reducing the risk of toxicity.

Therefore, there is a need for a method of enhancing the bioavailability of existing pharmaceutical drugs. Pharmaceutical compounds or pharmaceutical compositions having enhanced bioavailability would be able to be administered at a lower concentration, thereby decreasing the risk of toxicity or adverse side effects caused by administration of the drug.

Medicinal mushrooms and other natural products have been used to treat various diseases and infections for hundreds of years around the world. Today, medicinal mushrooms are also used to treat many types of diseases including lung diseases and many types of cancer around the world. For more than 30 years, medicinal mushrooms have been approved as an addition to standard cancer treatments in Japan and China. In these countries, mushrooms have been used safely for a long time, either alone or combined with radiation or various chemotherapeutics. More than 100 different types of mushrooms are used to treat cancer in Asia, including Ganoderma lucidum (relish), Trametes versicolor or Curious versicolor (turkey tail), Lentinus edodes (shiitake), and Grifola frondosa (maitake). Since many mushrooms are edible and non-toxic, they provide the possibility that one or more of their constituent compounds may provide an effective, non-toxic treatment for cancer.

Of particular interest is how mushrooms affect the immune system and if they stop or slow the growth of tumors or kill tumor cells. It is thought that certain chemical compounds, such as polysaccharides (beta-glucans), triterpenes, alkaloids and other forms of biomolecules strengthen the immune system to fight cancer directly as well as exert direct anticancer functions on the cancer cells themselves. Their potential uses, both individually and as adjuncts to cancer therapy have emerged. Mushrooms are also known to complement chemotherapy and radiation therapy by countering the side-effects of cancer, such as nausea, bone marrow suppression, anemia, and lowered resistance. Recently, a number of bio-active molecules, including anti-tumor agents, have been identified from various mushrooms. These bioactive molecules include polysaccharides, proteins, fats, ash, glycosides, alkaloids, volatile oils, tocopherols, phenolics, flavonoids, carotenoids, folates, ascorbic acid enzymes and organic acids. Polysaccharides, such as Beta-D-glucan, are the widest known mushroom-derived compounds with anti-cancer and immunomodulating properties. For example, polysaccharide K (PSK), a protein-bound polysaccharide found in turkey tail mushrooms, is an approved mushroom product used to treat cancer in Japan and has been studied as an adjuvant therapy in the treatment of gastric (stomach) cancer, breast cancer, colorectal cancer and lung cancer.

A dietary supplement prepared from Trametes versicolor the turkey tail mushroom, has been shown to reduce the growth of hormone responsive prostate cancer LNCaP cells. A crude extract of T. versicolor has been shown to inhibit growth in a number of human cancer cell lines, including gastric cancer (7907), lung cancer (SPC), leukemia (MCL) and lymphoma (SLY). The polysaccharide of this mushroom has been shown to inhibit the proliferation of human hepatoma cancer (QCY) cells in vitro and in vivo, which occurred with apoptosis and a decrease in the expression of the cell cycle-related genes, p53, Bcl-2 and Fas.

The genus Ganoderma, commonly known as Reishi or Lingzhi, has traditionally been administered throughout Asia as an anti-cancer agent for centuries. Extracts of G. lucidum have been shown to decrease the viability of human gastric carcinoma cells. Ganoderic acid T (GA-T) has been shown to inhibit tumor invasion and metastasis in human colon cancer cells lines, while other ganoderic acids, including (GA-Me, GA-Mf, GA-S) have been shown to be cytotoxic to human colon carcinoma cells and to decrease cell population growth in human carcinoma cell lines. A native glycopeptide, LZ-D-4, purified from the fruiting bodies of G. lucidum and its sulfated derivative, LZ-D, showed anti-tumor activity in vitro against mouse lymphocytic leukemia.

A d-glucan purified from Grifola frondosa, known as the dancing mushroom or Maitake, has been shown to enhance the efficacy of cisplatin, checking the decrease in the number of immunocompetent cells, namely macrophages, DCs and NK cells in cisplatin-treated mice. A chemically-sulfated polysaccharide (S-GAP-P) derived from a water insoluble polysaccharide of G. frondosa, has been shown to have anti-cancer effects when used in combination with 5-fluorouracil (5-FU) in human carcinoma cells, inhibiting cell growth and inducing cell apoptosis. A polysaccharide-peptide, GFPPS1b, isolated from cultured mycelia of G. frondose was shown to have anti-tumor activity, which inhibited the proliferation of human gastric adenocarcinoma cells. The cells succumbed to apoptosis, which was associated with a drop in mitochondrial transmembrane potential, up-regulation of Bax, down regulation of Bsl-2 and activation of caspase-3.

Several different additional mushroom types have been the subject of anti-cancer studies. A water and ethanol extract of one of these, the Chaga mushroom (Inonotus obliquus), has been shown to induce apoptosis in human colon cancer (DLD-1) cells by prevention of reactive oxygen species (ROS)-induced tissue damage, among other functions. A water extract of Chaga was also shown to arrest the cell cycle at the Go/G1 phase in B16-F10 murine melanoma cells, causing not only apoptosis, but also induced cell differentiation. These effects were associated with the down-regulation of pRb, p53 and p27 expression levels, and further shows that the Chaga extract resulted in a Go/G₁ cell cycle arrest with reduction of cyclin E/D1 and Cdk 2/4 expression levels. Furthermore, the anti-tumor effect of Chaga extract was assessed in vivo in Balb/c mice. Intraperitoneal administration of Chaga extract significantly inhibited the growth of tumor mass in B16-F10 cells implanted in mice, resulting in 3-fold inhibition at a dose of 20 mg/kg/day for 10 days. The ethanolic extract of Sclerotium and fruiting body of Chaga elicited significant anti-tumor activity 74.6% and 44.2% respectively.

The pentacyclic triterpenoid betulinic acid occurs naturally in Chaga and has been the subject of a number of studies for its anti-cancer properties due to its anti-tumoral activity and the ability to overcome resistance by inducing apoptosis in a variety of human cancers. Its selective cytotoxicity against cancer was first described in human melanoma both in vitro and in vivo in 1995. Since then, betulinic acid has been reported to be effective on a number of human cancers, including lung cancers, colon, prostate, and ovary. One study has shown that normal cells remain unaffected by betulinic acid treatment. Betulinic acid has also been applied in vitro in childhood cancers, viz. medulloblastoma, glioblastoma, Ewing sarcoma, neuroblastoma, and leukemia. Accumulated experimental evidence shows that betulinic acid treatment results in morphological change in sensitive cells, such as cell shrinkage, DNA fragmentation, nuclear condensation, and membrane blebbing. While the exact molecular mechanism underlying betulinic acid-induced apoptosis remains unclear, several studies suggest that the proteolytic cleavage of caspases, the activation of the MAP kinase cascade, the modulation of NF-KB signaling, the generation of reactive oxygen species, and the inhibition of topoisomerase I may all be contributing processes. Along with these functions, betulinic acid has also been found to reactivate the mitochondria, and induce apoptosis by releasing cytochrome C, a signaling protein that induces apoptosis naturally in response to irreparable cellular damage.

There is a need to further explore the potential benefits of Chaga mushrooms, particularly the potential oncological benefits. One limitation discovered in the continuing research of betulinic acid, and discussed openly among many research groups, is the poor bioavailability of betulinic acid. Scientists have speculated that this low bioavailability is caused by the poor water solubility of betulinic acid. The low bioavailability of betulinic acid severely limits practical applications of betulinic acid as a therapeutic agent. Various attempts to overcome this low bioavailability have been tried and have included self-nanoemulsifying drug delivery systems and spray gun technologies, but none have provided a practical solution.

In addition to betulinic acid, other naturally-occurring chemicals exist in Chaga mushrooms. Therefore, what is needed is a therapeutic component, isolated from the Chaga mushroom having sufficient bioavailability to provide a therapeutic effect when administered to humans and animals.

### SUMMARY

Methods for increasing the bioavailability of triterpene chemical compounds are described herein. Also provided are triterpene compounds having enhanced bioavailability produced by the methods. The bioavailable triterpene compounds are useful in pharmaceutical compositions for the treatment of diseases, such as cancer. In addition, a triterpene compound having enhanced bioavailability is combined with a therapeutic drug to provide simultaneous administration. Alternatively, the triterpene and therapeutic drug are administered sequentially. Furthermore, the triterpene compound having enhanced bioavailability is reacted with a therapeutic drug to form a triterpene-drug complex.

The therapeutic drug is a human or animal drug such as a chemotherapeutic agent, analgesic, neurotransmitter, opioid, hormones or corticosteroid. The therapeutic drug benefits from the enhanced bioavailability of the triterpene in that the bioavailable triterpene enhances the bioavailability of the therapeutic drug when administered before, after, simultaneously with, or complexed with administration of the therapeutic drug. In particular, a therapeutic drug that causes adverse side effects when administered to a subject is given in conjunction with administration of the triterpene having enhanced bioavailability. The bioavailable triterpene increases the bioavailability of the therapeutic drug so that less drug can be administered to achieve the desired therapeutic effect. The administration of a lower dose of drug thereby reduces the onset of adverse side effects.

One embodiment is a method for increasing the bioavailability of a triterpene chemical compound by utilizing the Maillard reaction to initiate a reducing sugar/amine group condensation reaction. A reducing sugar is combined with an amino acid and heated at an elevated temperature under vacuum for a sufficient amount of time to form one or more Maillard reaction products. The one or more Maillard reaction products are added to a triterpene compound, and the solution is brought to a boil under vacuum. The triterpene is esterified with a fatty acid under similar reaction conditions to produce an emulsion or micelles. The emulsion or micelles contain the triterpene, which has been chemically modified to exhibit increased bioavailability when compared with the bioavailability of the corresponding naturally-occurring, non-modified triterpene. In another embodiment, the triterpene is a pentacyclic triterpene, or phenolic, which is classified as a molecular structure containing at least one aromatic ring with at least one hydroxyl (—OH) group. In another embodiment, the triterpene is a pentacyclic triterpene, or phenolic, which is classified as a molecular structure containing at least one aromatic ring without a hydroxyl group, but with other functional groups such as cycloalkanes; cycloalkenes; cycloalkynes; arenes; styrenes; polycyclic aromatic hydrocarbons; heterocyclic ring structures that also contain a non-carbon element such as N, O, or S; and aromatic-amides, -amines-, -halides, -ketones, -aldehydes, -esters, -ethers, -acids, or nitro. In another embodiment, the esterification reaction is maintained under vacuum until the reaction has stopped to allow for the formation of stable products that remain intact and chemically active for a prolonged period of time.

Another embodiment is a bioavailable triterpene chemical compound. The triterpene is a reaction product produced by the method described herein for increasing the bioavailability of the triterpene, over the naturally-occurring triterpene, by reduction, esterification and emulsification or the bioavailable triterpene is synthesized. Optionally, the bioavailable triterpene is combined with a pharmaceutically-acceptable carrier to create a pharmaceutical composition.

Another embodiment is a method of treating a disease, such as cancer, by administering one or more of the triterpene chemical compounds having enhanced bioavailability or pharmaceutical compositions containing the chemical compounds having enhanced bioavailability to a human or animal.

Another embodiment is a method for increasing the bioavailability of a therapeutic drug by combining the therapeutic drug with a triterpene having enhanced bioavailability.

Another embodiment is a composition containing a bioavailable triterpene chemical compound in combination with a therapeutic drug. The combination is a mixture or is a reaction product in which the triterpene is joined to the therapeutic agent by an R group. In an embodiment, the R group is a saccharide, such as a sugar molecule, or a polysaccharide. Optionally, the bioavailable triterpene and therapeutic drug are combined with a pharmaceutically-acceptable carrier to create a pharmaceutical composition.

Another embodiment is a method of treating a disease, such as cancer, by administering to a human or animal one or more triterpene chemical compounds having enhanced bioavailability and administering one or more therapeutic drugs, sequentially or simultaneously in a mixture or complex.

Another embodiment is a method of treating a disease, such as cancer, by administering to a human or animal a pharmaceutical composition containing a mixture of one or more triterpene compounds having enhanced bioavailability in combination with one or more therapeutic drugs, in a pharmaceutically-acceptable carrier.

Another embodiment is a method of treating a disease, such as cancer, by administering to a human or animal a pharmaceutical composition containing a complex of one or more of the triterpene compounds having enhanced bioavailability in combination with one or more therapeutic drugs, in a pharmaceutically-acceptable carrier.

Another embodiment is a method for enhancing the bioavailability of a variety of bioactive chemicals, compounds or substances isolated from Chaga mushrooms. In accordance with the method, a reduction of Chaga mushroom is formed in a reduction solvent under pressure, an extraction of Chaga mushroom is formed in an extraction solvent, and the reduction and extraction are mixed together, or combined, in accordance with methods known to those skilled in the art to form a reduction and extraction mixture. The reduction and extraction mixture is then combined with an esterification agent. One embodiment of the esterification agent is a mixture containing proline, fructose, and a fatty acid such as a medium chain triglyceride (MCT) oil or sunflower lecithin, or other carbon chain fatty acids of various carbon chain length, with and without phospholipids, for the esterification of the reduction and extraction mixture of esterified compounds. Alternatively, sources of sugar esters capable of emulsion within otherwise non-miscible liquid compounds, or a predetermined amount of Manuka honey. The amount of esterification mixture added should be sufficient to cause esterification and facilitate emulsion at a temperature capable of inducing a reaction between the bioactive substances of the Chaga mushroom and the constituents of the esterification mixture, or Manuka honey, to form a miscible heterogenous mixture. This miscible heterogenous mixture is then combined with a medium chain triglyceride to produce a final emulsified solution containing chemical-modified compounds of the Chaga mushroom having enhanced bioavailability.

Another embodiment is a chemical compound. The chemical compound is isolated from the final solution or is synthesized and is optionally combined with a pharmaceutically-acceptable carrier to form a pharmaceutical composition.

Another embodiment is a method of treating a disease, such as cancer, by administering one or more of the chemical compounds having enhanced bioavailability or pharmaceutical compositions containing the chemical compounds having enhanced bioavailability to a human or animal.

The following are additional various concepts numbered for reference in other concepts.

Concept 1—A method of increasing the bioavailability of a triterpene by combining a reducing sugar with an amino acid, heating the combination at an elevated temperature under vacuum for a sufficient amount of time to form one or more Maillard reaction products, adding the one or more Maillard reaction products to a triterpene to produce a triterpene reaction mixture, boiling the reaction mixture under vacuum, and esterifying the reaction mixture with a fatty acid under to produce an emulsion, wherein the emulsion comprises a modified triterpene having increased bioavailability.

Concept 2—A bioavailable triterpene produced by the method of Concept 1.

Concept 3—The bioavailable triterpene of Concept 2, wherein the triterpene has one of the chemical structures of chemical structures 1-77 of the list of Chemical Structures of Triterpenes.

Concept 4—A bioavailable triterpene having the chemical formula of Formula 1 or Formula 2.

Concept 5—A bioavailable triterpene, wherein the triterpene has one of the chemical structures of chemical structures 1-77 of the List of Chemical Structures of Triterpenes.

Concept 6—A bioavailable triterpene, wherein the bioavailable triterpene is synthetically produced.

Concept 7—A pharmaceutical composition comprising the bioavailable triterpene of Concepts 2-6 in a pharmaceutically-acceptable carrier.

Concept 8—A method of treating a disease by administering the pharmaceutical composition of Concept 7 to a human or animal having the disease

Concept 9—The method of Concept 8, wherein the disease is cancer

Concept 10—A method for increasing the bioavailability of a therapeutic drug by combining the therapeutic drug with a bioavailable triterpene

Concept 11—A pharmaceutical composition containing a combination of a bioavailable triterpene and a therapeutic drug in a pharmaceutically-acceptable carrier

Concept 12—The method of Concept 10, wherein the bioavailable triterpene is the bioavailable triterpene of Concepts 2-6.

Concept 13—The pharmaceutical composition of Concept 12, wherein the bioavailable triterpene is produced by the method of Concept 1.

Concept 14—The pharmaceutical composition of Concept 13, wherein the bioavailable triterpene has one of the chemical structures of chemical structures 1-77 of the list of Chemical Structures of Triterpenes

Concept 15—The pharmaceutical composition of Concept 12, wherein the bioavailable triterpene has the chemical formula of Formula 1 or Formula 2.

Concept 16—The pharmaceutical composition of Concept 12, wherein the triterpene has one of the chemical structures of chemical structures 1-77 of the list of Chemical Structures of Triterpenes.

Concept 17—The pharmaceutical composition of Concept 12, wherein the bioavailable triterpene is synthetically produced.

Concept 18—The pharmaceutical composition of Concept 12 wherein the combination is a mixture of a bioavailable triterpene and a therapeutic drug in a pharmaceutically-acceptable carrier.

Concept 19—The pharmaceutical composition of Concept 18, wherein the bioavailable triterpene is produced by the method of Concept 1

Concept 20—The pharmaceutical composition of Concept 19, wherein the bioavailable triterpene has one of the chemical structures of chemical structures 1-77 of the list of Chemical Structures of Triterpenes

Concept 21—The pharmaceutical composition of Concept 18, wherein the bioavailable triterpene has the chemical formula of Formula 1 or Formula 2

Concept 22—The pharmaceutical composition of Concept 18, wherein the bioavailable triterpene has one of the chemical structures of chemical structures 1-77 of the list of Chemical Structures of Triterpenes

Concept 23—The pharmaceutical composition of Claim 18, wherein the bioavailable triterpene is synthetically produced

Concept 24—The pharmaceutical composition of Claim 12 wherein the combination is a complex of a bioavailable triterpene and a therapeutic drug in a pharmaceutically-acceptable carrier

Concept 25—The pharmaceutical composition of Concept 24, wherein the bioavailable triterpene chemical compound is complexed to the therapeutic drug with a saccharide or polysaccharide

Concept 26—The pharmaceutical composition of Concepts 24-25, wherein the bioavailable triterpene is produced by the method of Claim 1.

Concept 27—The pharmaceutical composition of Concept 26, wherein the bioavailable triterpene is a triterpene having one of the chemical structures of chemical structures 1-77 of the list of Chemical Structures of Triterpenes.

Concept 28—The pharmaceutical composition of Concepts 24-25, wherein the bioavailable triterpene has the chemical formula of Formula 1 or Formula 2.

Concept 29—The pharmaceutical composition of Concepts 24-25, wherein the triterpene has one of the chemical structures of chemical structures 1-77 of the list of Chemical Structures of Triterpenes.

Concept 30—The pharmaceutical composition of Concepts 24-25, wherein the bioavailable triterpene is synthetically produced.

The pharmaceutical composition of Concepts 11-30 wherein the therapeutic drug is a chemotherapeutic agent, analgesic, neurotransmitter, opioid, hormones or corticosteroid.

Concept 31—A method of treating a disease by administering the pharmaceutical composition of Concepts 11-30 to a human or animal having the disease

Concept 32—The method of Concept 31, wherein the disease is cancer.

Concept 33—A method of increasing the bioavailability of a bioactive Chaga mushroom isolate by forming a reduction of Chaga mushroom in a reducing solvent; forming an extraction of Chaga mushroom in an extraction solvent; mixing the reduction of Chaga mushroom with the extraction of Chaga mushroom, and isolating a bioactive chemical compound, wherein the compound has increased bioavailability when compared with a naturally-occurring bioactive Chaga mushroom isolate.

Concept 34—A bioactive chemical isolate of a Chaga mushroom having enhanced bioavailability prepared by forming a mixture of a Chaga mushroom reduction and a Chaga mushroom extraction with an esterification mixture comprising proline, fructose and a fatty acid, and an emulsifying sugar ester capable of emulsion within otherwise non-miscible liquids to facilitate emulsion at a temperature that results in a reaction between the Chaga mushroom reduction and Chaga mushroom extraction mixture with the esterification mixture to produce a reaction mixture, and combining the reaction mixture with a medium chain triglyceride oil to make the Chaga mushroom isolate having enhanced bioavailability.

Concept 35—The method of Concept 35, wherein the esterification mixture is a Manuka honey

Concept 36—A method of making a Chaga mushroom isolate having enhanced bioavailability by preparing a Chaga mushroom reduction in a reduction solvent under high pressure; preparing a Chaga mushroom extraction in an extraction solvent; mixing the Chaga reduction with the Chaga extraction; reacting the Chaga reduction and Chaga extraction mixture with an esterification mixture comprising proline, fructose and a fatty acid, and an emulsifying sugar ester capable of emulsion within otherwise non-miscible liquids to facilitate emulsion at a temperature that results in a reaction between the Chaga mushroom reduction and Chaga mushroom extraction mixture with the esterification mixture to produce a reaction mixture and combining the reaction mixture with medium chain triglyceride oil to make the Chaga mushroom isolate having enhanced bioavailability when compared with a naturally-occurring bioactive Chaga mushroom isolate.

Concept 37—The method of Concept 38, wherein the esterification mixture is a Manuka honey.

### BRIEF DESCRIPTION OF THE DRAWINGS

The invention may be more completely understood in consideration of the following detailed description of various embodiments of the invention in connection with the accompanying drawings, in which:

FIG. 1 depicts the chemical structures of the starting materials for the series of reactions that result in the production of the bioavailable triterpene as described herein. Proline is shown in this figure as an exemplary amino acid; glucose is shown as an exemplary reducing sugar, i.e. glycosidic bond, (fructose is just as viable a reducing sugar); trametanolic acid is shown as an exemplary triterpene (however, all the triterpenes shown in the chemical structures reproduced herein and listed as structures extracted from Chaga are equally viable); and decanoic acid is shown as an example of a fatty acid participating in the esterification reaction (however, any fatty acid or surfactant could be substituted).

FIG. 2 is a drawing of the chemical structure of an exemplary bioavailable triterpene, which was derived from trametanolic acid using the method described herein, and a drawing of the chemical structure of a pharmaceutical anti-cancer drug, Doxorubicin. This is an example of the A+B concept wherein A is the pharmaceutical drug (Doxorubicin) and B is the bioavailable triterpene. When administered to a subject, this combination increases the bioavailability of the drug, permitting a lower dose of drug to be administered to achieve the therapeutic effect, thereby reducing the risk of drug toxicity.

FIG. 3 is a drawing of the chemical structure of a complex of an exemplary bioavailable triterpene, which was derived from trametanolic acid using the method described herein, and a pharmaceutical anti-cancer drug, Doxorubicin. This is an example of the A-R-B concept wherein A is the pharmaceutical drug (Doxorubicin), B is the bioavailable triterpene and R is a saccharide. When administered to a subject, this complex increases the bioavailability of the drug, permitting a lower dose of drug to be administered to achieve the therapeutic effect, thereby reducing the risk of drug toxicity.

FIG. 4 is a drawing of the chemical structure of an exemplary bioavailable triterpene, which was derived from trametanolic acid using the method described herein, and a drawing of the chemical structure of a pharmaceutical steroid drug, Prednisone. This is an example of the A+B concept wherein A is the pharmaceutical drug (Prednisone) and B is the bioavailable triterpene. When administered to a subject, this combination increases the bioavailability of the drug, permitting a lower dose of drug to be administered to achieve the therapeutic effect, thereby reducing the risk of drug toxicity.

FIG. 5 is a drawing of the chemical structure of a complex of an exemplary bioavailable triterpene, which was derived from trametanolic acid using the method described herein, and a pharmaceutical steroid drug, Prednisone. This is an example of the A-R-B concept wherein A is the pharmaceutical drug (Prednisone), B is the bioavailable triterpene and R is a saccharide. When administered to a subject, this complex increases the bioavailability of the drug, permitting a lower dose of drug to be administered to achieve the therapeutic effect, thereby reducing the risk of drug toxicity.

FIG. 6 is a drawing of the chemical structure of an exemplary bioavailable triterpene, which was derived from trametanolic acid using the method described herein, and a drawing of the chemical structure of a pharmaceutical anti-cholesterol drug, Lovastatin. This is an example of the A+B concept wherein A is the pharmaceutical drug (Lovastatin) and B is the bioavailable triterpene. When administered to a subject, this combination increases the bioavailability of the drug, permitting a lower dose of drug to be administered to achieve the therapeutic effect, thereby reducing the risk of drug toxicity.

FIG. 7 is a drawing of the chemical structure of a complex of an exemplary bioavailable triterpene, which was derived from trametanolic acid using the method described herein, and a pharmaceutical anti-cholesterol drug, Lovastatin. This is an example of the A-R-B concept wherein A is the pharmaceutical drug (Lovastatin), B is the bioavailable triterpene and R is a saccharide. When administered to a subject, this complex increases the bioavailability of the drug, permitting a lower dose of drug to be administered to achieve the therapeutic effect, thereby reducing the risk of drug. Note that the peroxide linkage may allow for much lower doses while maintaining efficacy.

FIG. 8 is a drawing of the chemical structure of an exemplary bioavailable triterpene, which was derived from trametanolic acid using the method described herein, and a drawing of the chemical structure of a pharmaceutical antidepressant medication, the Serotonin-norepinephrine re-uptake inhibitor (SNRI), Venlafixine. This is an example of the A+B concept wherein A is the pharmaceutical drug (Venlafixine, also known as Effexor®) and B is the bioavailable triterpene. When administered to a subject, this combination increases the bioavailability of the drug, permitting a lower dose of drug to be administered to achieve the therapeutic effect, thereby reducing the risk of drug toxicity.

FIG. 9 is a drawing of the chemical structure of a complex of an exemplary bioavailable triterpene, which was derived from trametanolic acid using the method described herein, and a pharmaceutical antidepressant medication, the Serotonin-norepinephrhine re-uptake inhibitor (SNRI), Venlafixine. This is an example of the A-R-B concept wherein A is the pharmaceutical drug (Venlafixine, also known as Effexor®), B is the bioavailable triterpene and R is a saccharide. When administered to a subject, this complex increases the bioavailability of the drug, permitting a lower dose of drug to be administered to achieve the therapeutic effect, thereby reducing the risk of drug. Note that the peroxide linkage may allow for much lower doses while maintaining efficacy.

FIG. 10 is a drawing of the chemical structure of an exemplary bioavailable triterpene, which was derived from trametanolic acid using the method described herein, and a drawing of the chemical structure of a pharmaceutical analgesic medication, the non-steroidal anti-inflammatory (NSAID) drug, Acetaminophen. This is an example of the A+B concept wherein A is the pharmaceutical drug (Acetaminophen, also known as Tylenol®) and B is the bioavailable triterpene. When administered to a subject, this combination increases the bioavailability of the drug, permitting a lower dose of drug to be administered to achieve the therapeutic effect, thereby reducing the risk of drug toxicity.

FIG. 11 is a drawing of the chemical structure of a complex of an exemplary bioavailable triterpene, which was derived from trametanolic acid using the method described herein, and a pharmaceutical analgesic medication, the non-steroidal anti-inflammatory (NSAID) drug, Acetaminophen. This is an example of the A-R-B concept wherein A is the pharmaceutical drug (Acetaminophen, also known as Tylenol®), B is the bioavailable triterpene and R is a saccharide. When administered to a subject, this complex increases the bioavailability of the drug, permitting a lower dose of drug to be administered to achieve the therapeutic effect, thereby reducing the risk of drug. Note that the peroxide linkage may allow for much lower doses while maintaining efficacy.

FIG. 12 provides chemical structures of bioactive compounds isolated or derived from Chaga mushrooms. These bioactive chemicals, in their naturally-occurring form, exhibit poor availability when administered to humans and other animals. The methods provided herein are useful for enhancing the bioavailability of such components

FIGS. 13A, 13B, 13C, and 13D are enlarged quadrants of FIG. 1, in that they combine to create FIG. 1. They are included to assure sufficient detail and disclosure. FIG. 13A is the top left quadrant of FIG. 12. FIG. 13B is the top right quadrant of FIG. 12. FIG. 13C is the bottom left quadrant of FIG. 12. FIG. 13D is the bottom right quadrant of FIG. 12.

FIG. 14 shows the chemical reaction synthesis pathway for four different products all as a result of the proposed theoretical reaction. Compounds 1-4 show the possible reaction and product outcomes of betulinic acid (BA) and methylglyoxal (MGO) in an isolated environment. Compounds 5-8 show the potential product outcomes from the proposed reaction between BA, MGO and the fatty acids present in MCT oil described above. Compounds 9-12 show the potential product outcomes from the proposed reaction between betulinic acid, proline, lecithin and fructose. It may be seen that Applicant's pending PCT/ULS2024/027210, filed May 1, 2024, incorporated by reference herein in its entirety, includes a FIG. 58 which corresponds to this FIG. 14. FIGS. 44-55 in PCT/US2024/027210 and the accompanying description in PCT/US2024/027210 are noted as portions of and related to FIG. 14, and the PCT description of these FIGS. 44-55 is again incorporated herein to describe the processes therein.

FIG. 15 is a chemical structure of MH CHEMSTRUCTURES 01, which is a list of molecular compounds naturally occurring in Manuka Honey. This also includes macromolecules i.e. proteins, lipids, carbohydrates and minerals including minerals classified as antioxidant minerals and ionic.

FIG. 16 is a chemical structure of MH CHEMSTRUCTURES 02 is a similar list of minerals naturally occurring in Manuka Honey.

FIG. 17 is a chemical structure of NIH CHEMSTRUCTURES 03 is a similar list of macromolecules naturally occurring in Manuka Honey.

FIG. 18 is a chemical structure of MH CHEMSTRUCTURES 04 is a similar list of macromolecules naturally occurring in Manuka Honey.

FIG. 19A shows the top of a page of notes and illustrates some potential reaction sites of bioactive constituents found in chaga mushrooms.

FIG. 19B shows the bottom of a page of notes. This shows some potential reaction sites of bioactive constituents found in chaga mushrooms.

FIG. 20 is a chemical structure of NOTES—MH CHEMSTRUCTURES. This shows some potential reaction sites of bioactive constituents found in Manuka honey.

FIG. 21 is a chemical structure of Formula 1.

FIG. 22 is a chemical structure of Formula 2.

FIG. 23 is a bar graph showing that Mytulin Restore increases the mitochondrial capacitance—there is a change (increase) in uptake values of Tetramethylrhodamine, Methyl Ester, Perchlorate (TMRM).

FIG. 24 is a line graph showing that the Mytulin Restore increases the efficiency of the mitochondrial membrane to maintain the charge gradient. The increased change in differential in depolarized mitochondrial membrane reduces ROS signaling/Proton leak.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

Described herein are methods for enhancing the bioavailability of chemical compounds. Also provided are bioavailable chemical compounds produced by the methods. Also provided are chemical compounds having enhanced bioavailability. Also provided are pharmaceutical compositions containing one or more of the bioavailable chemical compounds described herein.

Also provided is a method for enhancing the bioavailability of one or more therapeutic or pharmaceutical drugs. Also provided are pharmaceutical compositions containing one or more of the bioavailable chemical compounds described herein and one or more pharmaceutical drug. Also provided is a complex of a bioavailable chemical compound linked to a therapeutic or pharmaceutical drug and a method of making the complex. The bioavailable chemical compounds described herein, alone or in combination with or complexed with, a therapeutic or pharmaceutical drug, are useful in pharmaceutical compositions for the treatment of a disease or disorder.

In an embodiment, methods for enhancing the bioavailability of triterpene chemical compounds are provided. Also provided are bioavailable triterpenes produced by the methods. Also provided are triterpene compounds having enhanced bioavailability. Also provided are pharmaceutical compositions containing one or more of the bioavailable chemical compounds described herein.

Another embodiment is a method for enhancing the bioavailability of one or more therapeutic or pharmaceutical drugs by combining the drug with one or more of the bioavailable triterpenes described herein. Also provided are pharmaceutical compositions containing one or more of the bioavailable triterpenes described herein and one or more therapeutic or pharmaceutical drugs. In another embodiment, a triterpene compound having enhanced bioavailability is combined with a therapeutic drug to provide simultaneous administration. Alternatively, the triterpene and therapeutic drug are administered sequentially. Furthermore, a complex of the bioavailable triterpene having enhanced bioavailability linked to a therapeutic or pharmaceutical drug to form a triterpene-drug complex. The bioavailable triterpene compounds described herein, alone or in combination with or complexed with a pharmaceutical drug, are useful in pharmaceutical compositions for the treatment of one or more diseases or disorders.

Triterpenes are a class of terpenes composed of six isoprene units with the molecular formula C₃₀H₄₈. Terpenes have the general formula (C₅H₈)ₙ and are known as biosynthetic building blocks. It follows that triterpenes contain three terpene units. Animals, plants and fungi all produce triterpenes. Although sometimes used interchangeably with the term terpene, a terpenoid is a modified terpene that contains additional functional groups. Chemical structure of a listing of triterpenes that can be modified as described herein to enhance their bioavailability are provided below as chemical structures 1-77. It will be understood by those skilled in the art that other triterpenes exist and could be used in the methods and compositions described herein. Preferred triterpenes to be modified to enhance bioavailability are those having superior stability. Stability, as used in this context, means that the chemical structures have the capacity to withstand the environment needed for the reactions to occur to form the triterpene having the enhanced bioavailability described herein. Such compounds can withstand the rigors of heat and pressure needed for those well-known reactions to proceed without breaking down. Generally, larger chemical structures are preferred. Chemical structures having additional structural groups also contribute to stability. Exemplary chemical structures most likely to be more stable are those such as, but not limited to, chemical structures 1-33 below.

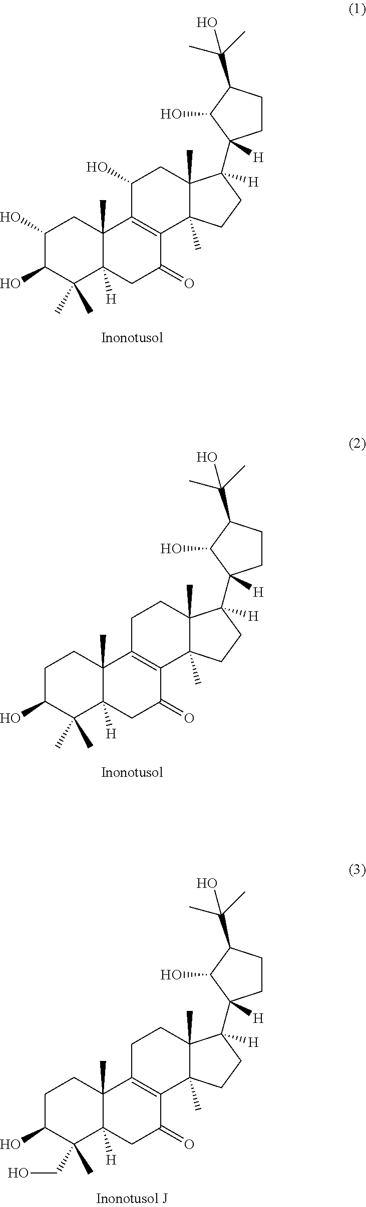

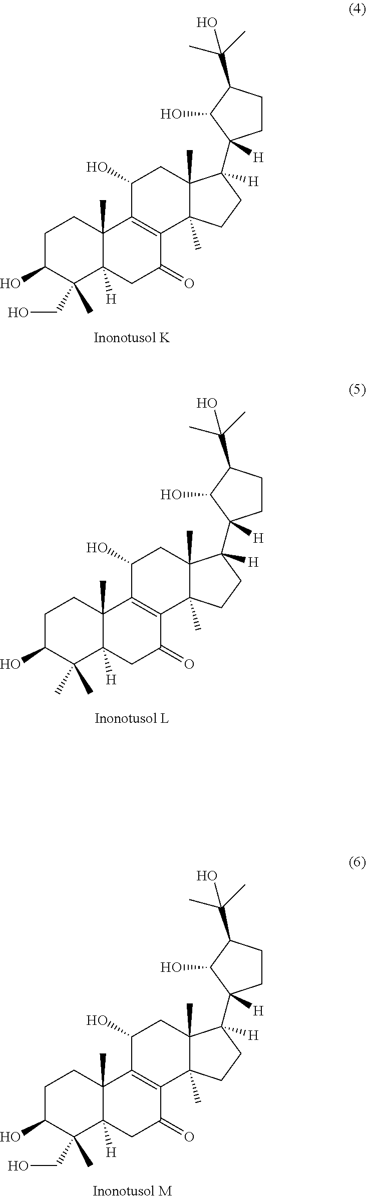

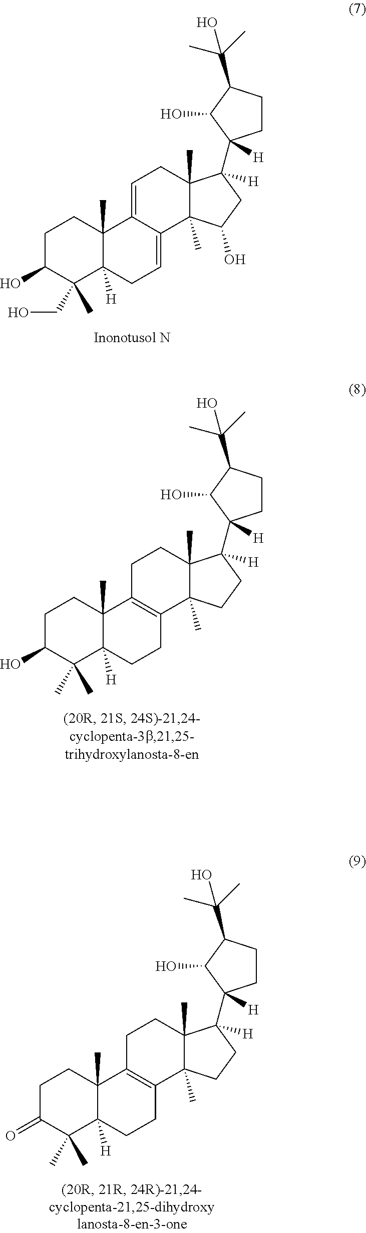

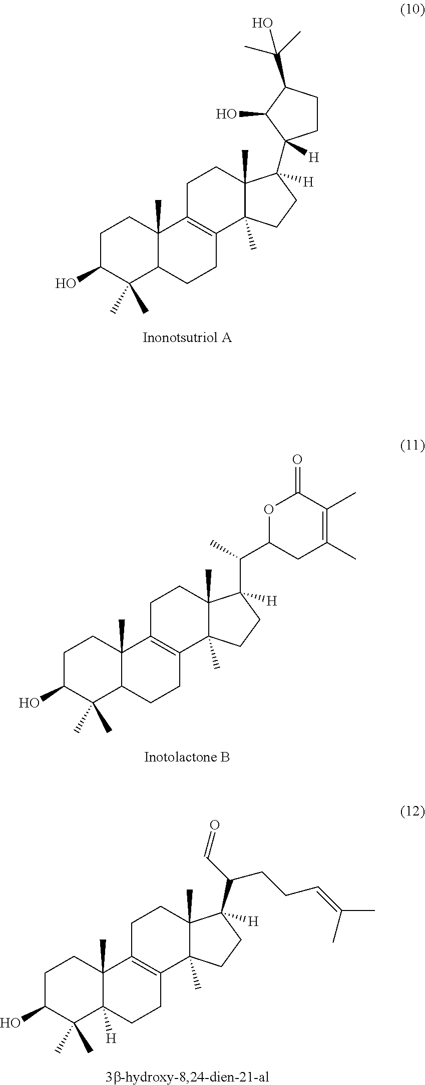

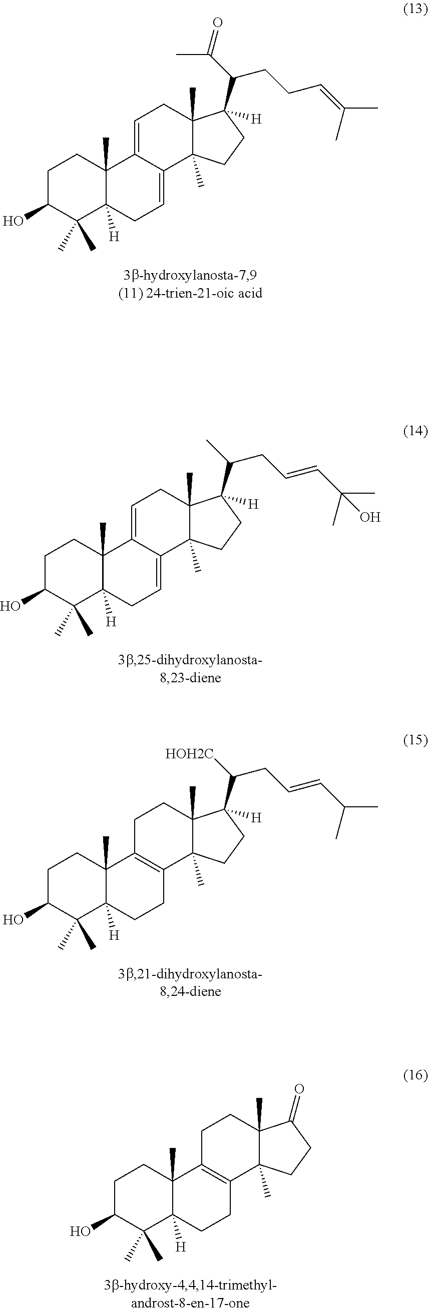

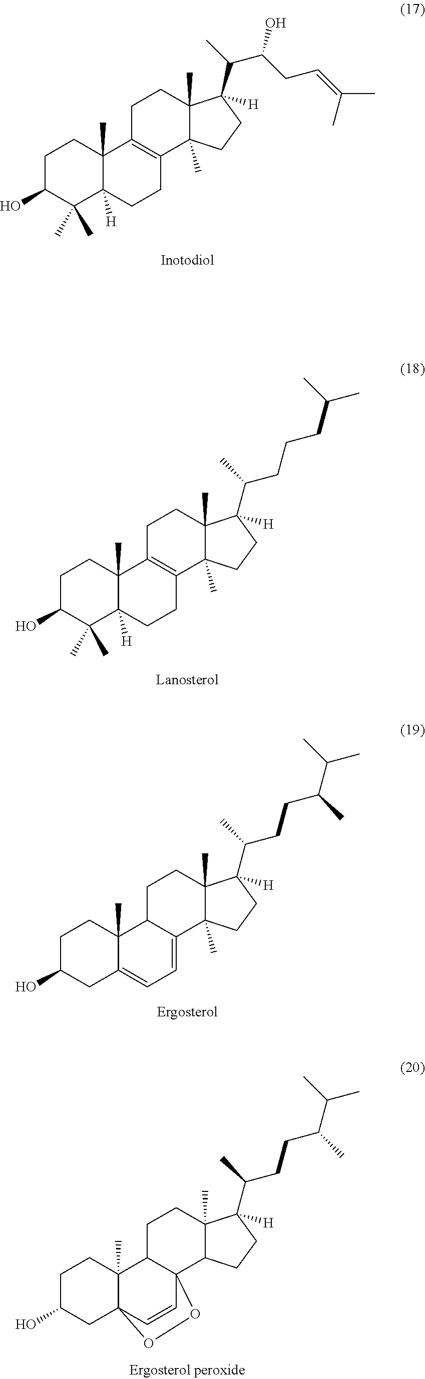

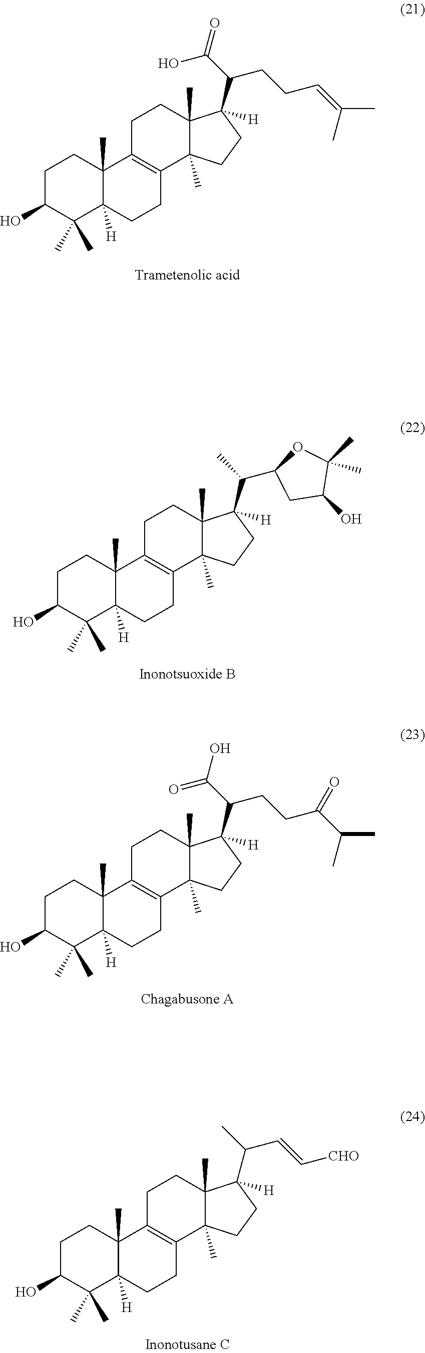

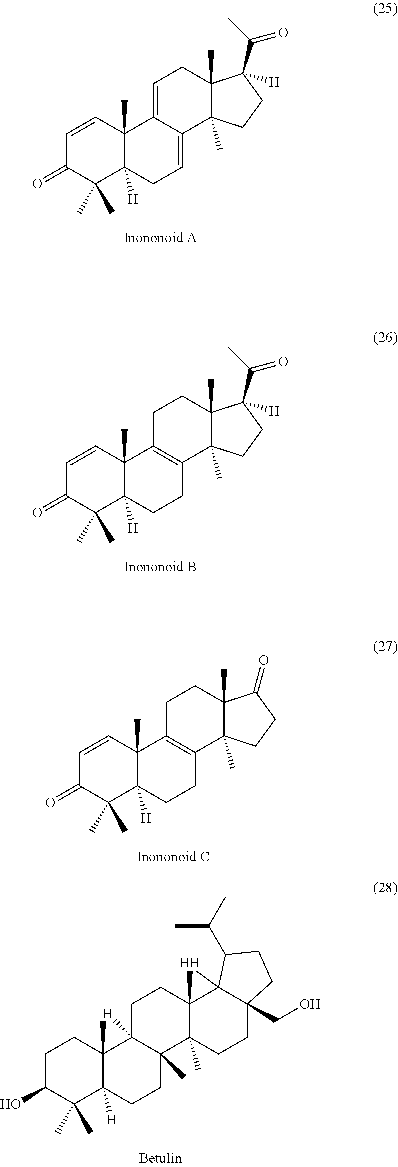

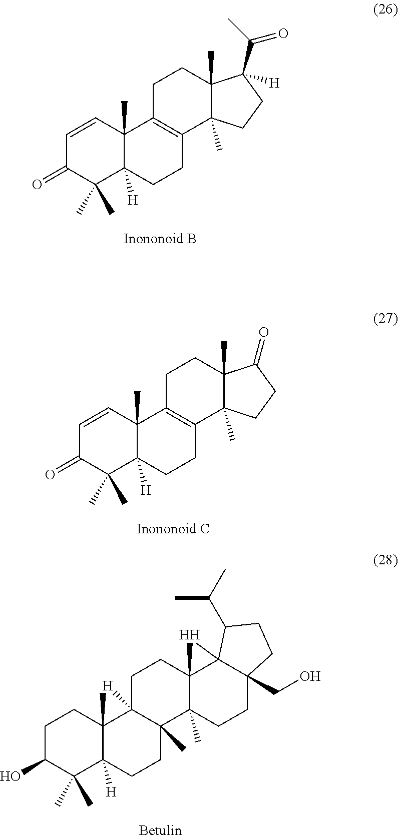

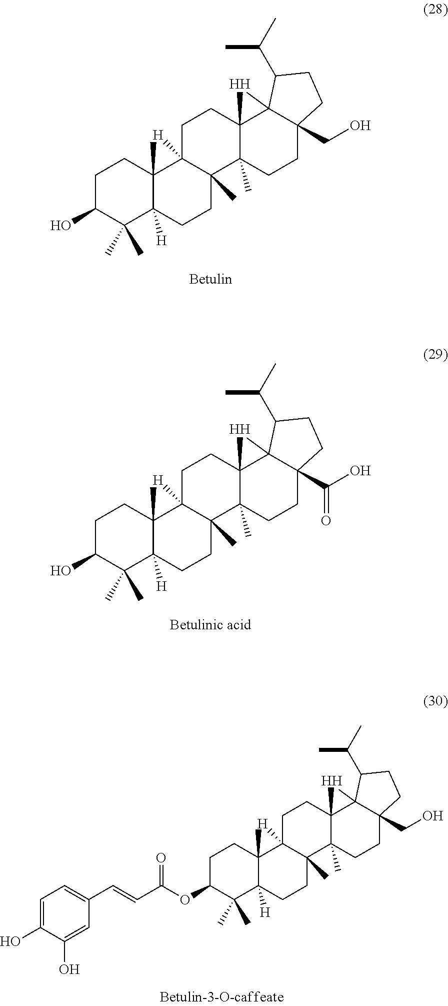

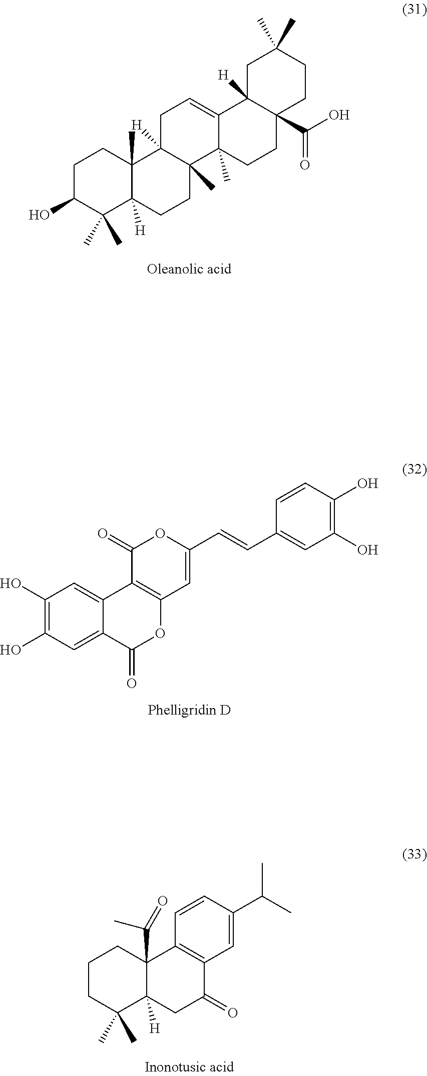

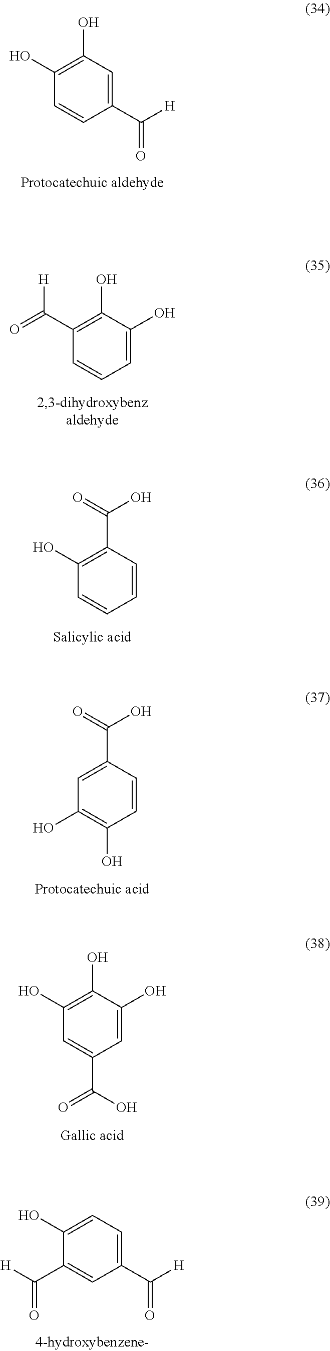 [SMILES: O=Cc1ccc(O)c(O)c1 ; O=Cc1cccc(O)c1O ; O=C(O)c1ccccc1O ; O=C(O)c1ccc(O)c(O)c1 ; O=C(O)c1cc(O)c(O)c(O)c1 ; O=Cc1ccc(O)c(C=O)c1]

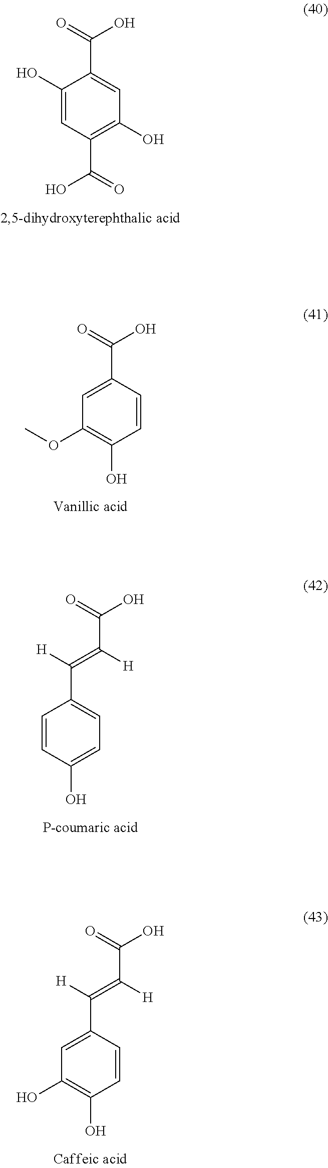

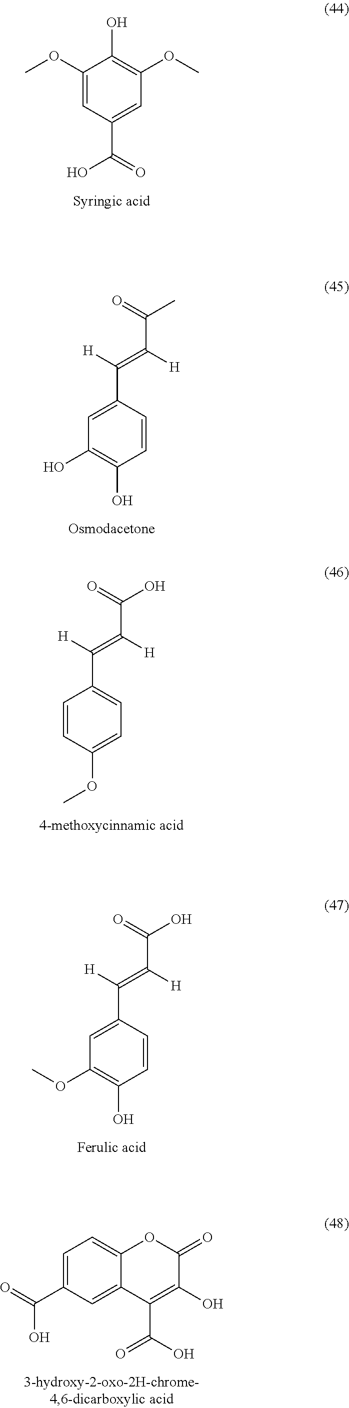

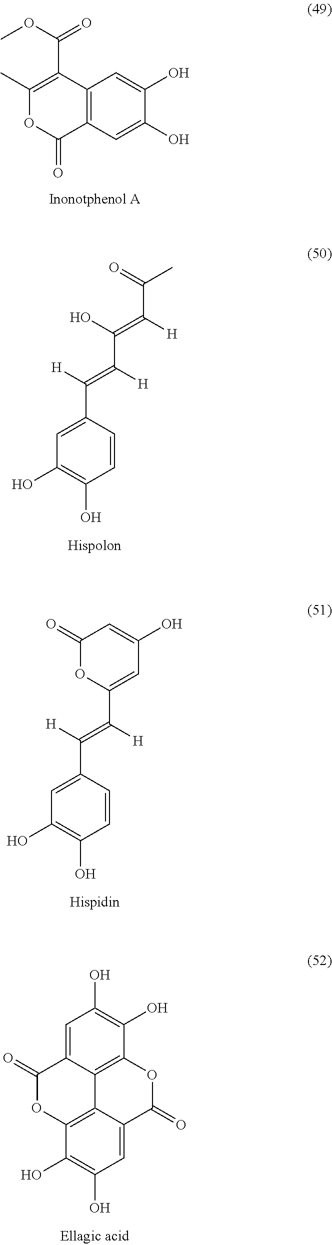 [SMILES: COC(=O)c1c(C)oc(=O)c2cc(O)c(O)cc12 ; CC(=O)/C=C(O)/C=C/c1ccc(O)c(O)c1 ; O=c1cc(O)cc(/C=C/c2ccc(O)c(O)c2)o1 ; O=c1oc2c(O)c(O)cc3c(=O)oc4c(O)c(O)cc1c4c23]

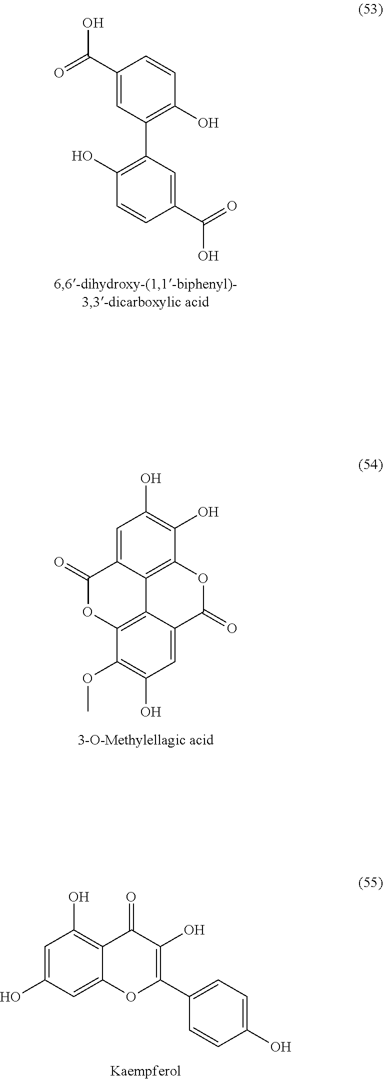 [SMILES: O=C(O)c1ccc(O)c(-c2cc(C(=O)O)ccc2O)c1 ; COc1c(O)cc2c(=O)oc3c(O)c(O)cc4c(=O)oc1c2c34 ; O=c1c(O)c(-c2ccc(O)cc2)oc2cc(O)cc(O)c12]

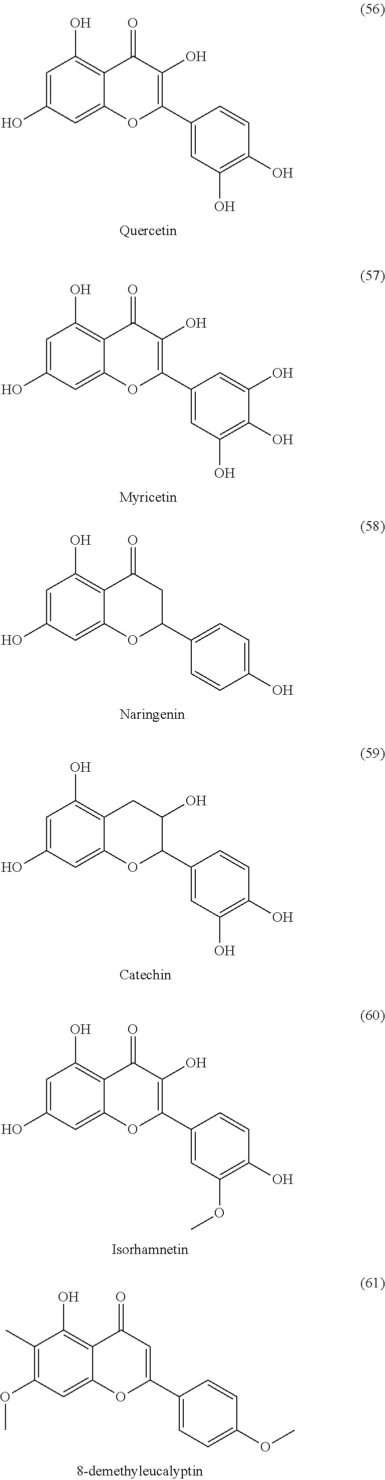 [SMILES: O=c1c(O)c(-c2ccc(O)c(O)c2)oc2cc(O)cc(O)c12 ; O=c1c(O)c(-c2cc(O)c(O)c(O)c2)oc2cc(O)cc(O)c12 ; O=C1CC(c2ccc(O)cc2)Oc2cc(O)cc(O)c21 ; Oc1cc(O)c2c(c1)OC(c1ccc(O)c(O)c1)C(O)C2 ; COc1cc(-c2oc3cc(O)cc(O)c3c(=O)c2O)ccc1O ; COc1ccc(-c2cc(=O)c3c(O)c(C)c(OC)cc3o2)cc1]

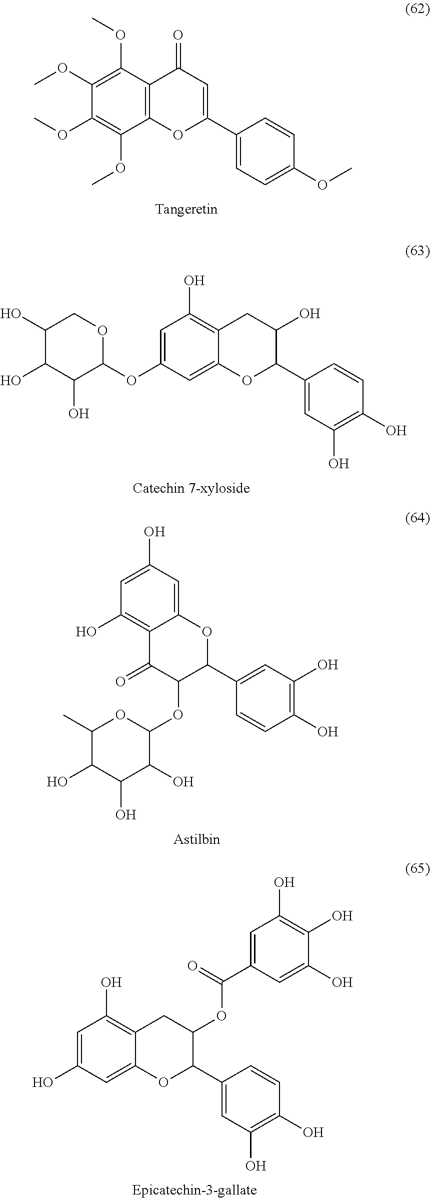

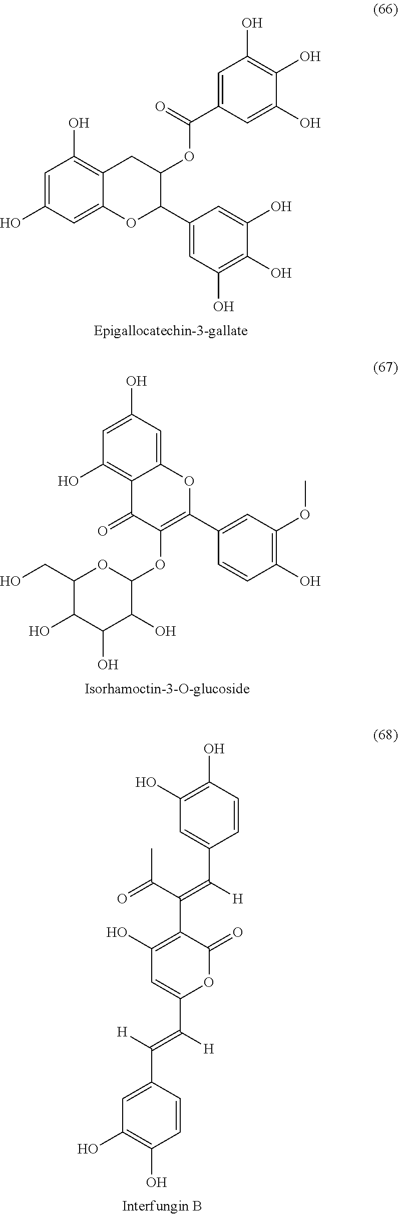 [SMILES: O=C(OC1Cc2c(O)cc(O)cc2OC1c1cc(O)c(O)c(O)c1)c1cc(O)c(O)c(O)c1 ; COc1cc(-c2oc3cc(O)cc(O)c3c(=O)c2OC2OC(CO)C(O)C(O)C2O)ccc1O ; CC(=O)/C(=C\c1ccc(O)c(O)c1)c1c(O)cc(/C=C/c2ccc(O)c(O)c2)oc1=O]

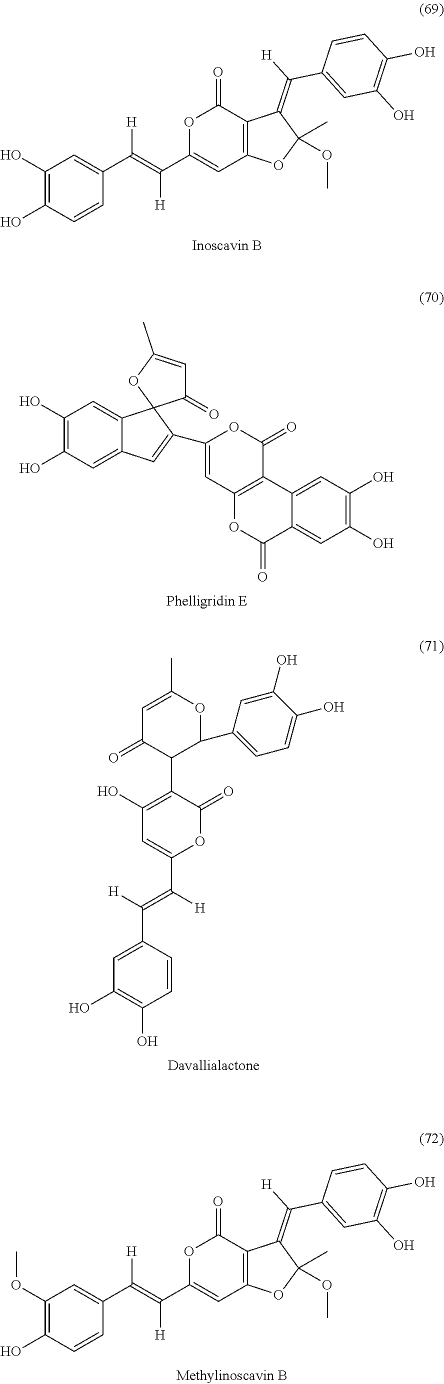 [SMILES: COC1(C)Oc2cc(/C=C/c3ccc(O)c(O)c3)oc(=O)c2/C1=C/c1ccc(O)c(O)c1 ; CC1=CC(=O)C2(O1)C(c1cc3oc(=O)c4cc(O)c(O)cc4c3c(=O)o1)=Cc1cc(O)c(O)cc12 ; CC1=CC(=O)C(c2c(O)cc(/C=C/c3ccc(O)c(O)c3)oc2=O)C(c2ccc(O)c(O)c2)O1 ; COc1cc(/C=C/c2cc3c(c(=O)o2)/C(=C/c2ccc(O)c(O)c2)C(C)(OC)O3)ccc1O]

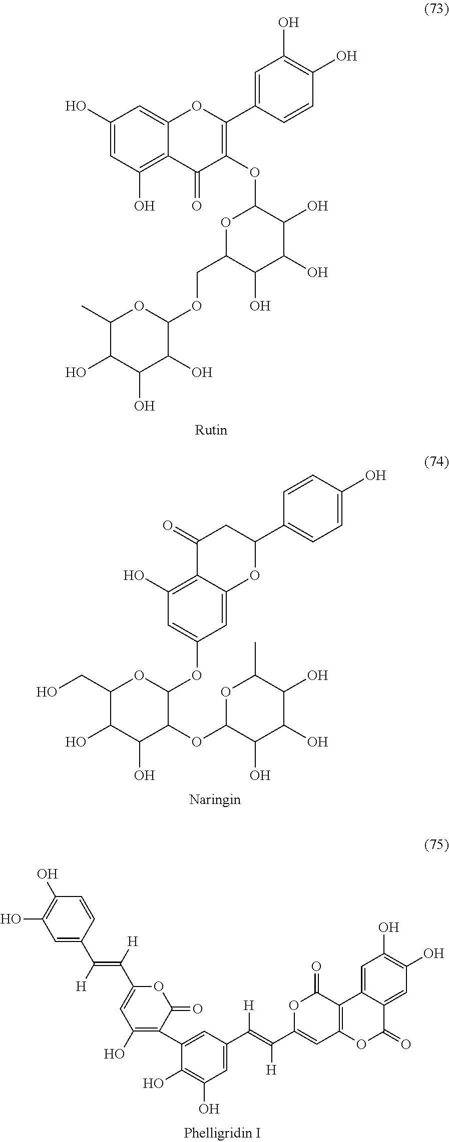 [SMILES: CC1OC(OCC2OC(Oc3c(-c4ccc(O)c(O)c4)oc4cc(O)cc(O)c4c3=O)C(O)C(O)C2O)C(O)C(O)C1O ; CC1OC(OC2C(Oc3cc(O)c4c(c3)OC(c3ccc(O)cc3)CC4=O)OC(CO)C(O)C2O)C(O)C(O)C1O ; O=c1oc(/C=C/c2ccc(O)c(O)c2)cc(O)c1-c1cc(/C=C/c2cc3oc(=O)c4cc(O)c(O)cc4c3c(=O)o2)cc(O)c1O]

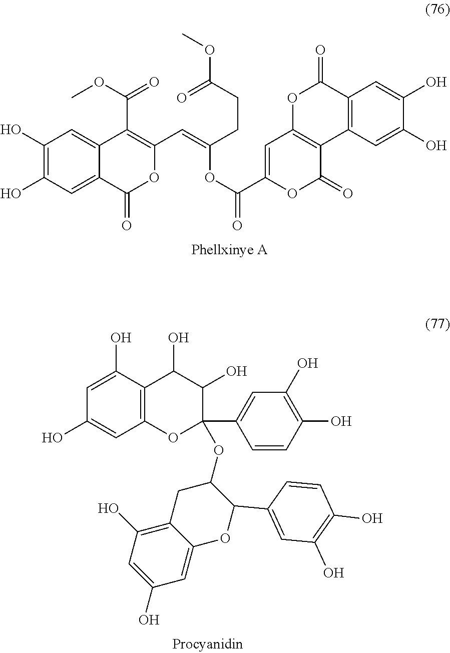 [SMILES: COC(=O)CC/C(=C/c1oc(=O)c2cc(O)c(O)cc2c1C(=O)OC)OC(=O)c1cc2oc(=O)c3cc(O)c(O)cc3c2c(=O)o1 ; Oc1cc(O)c2c(c1)OC(c1ccc(O)c(O)c1)C(OC1(c3ccc(O)c(O)c3)Oc3cc(O)cc(O)c3C(O)C1O)C2]

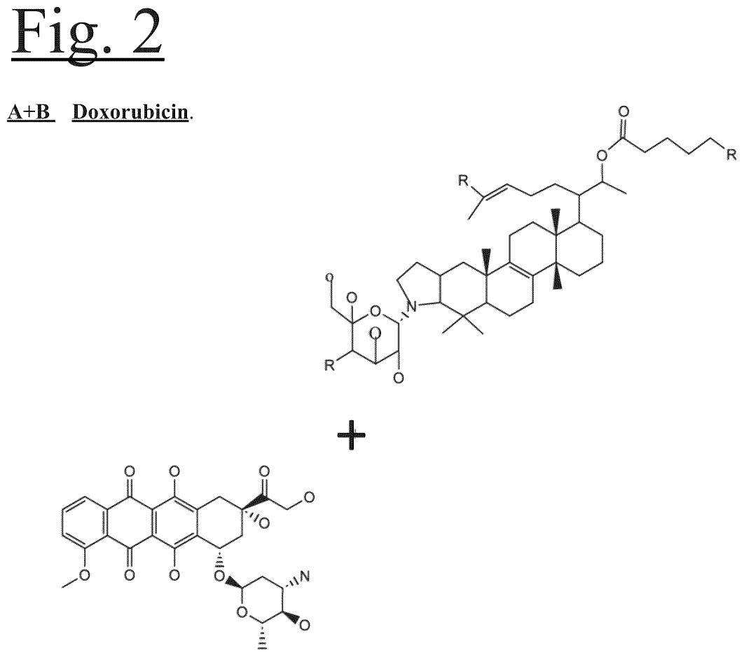

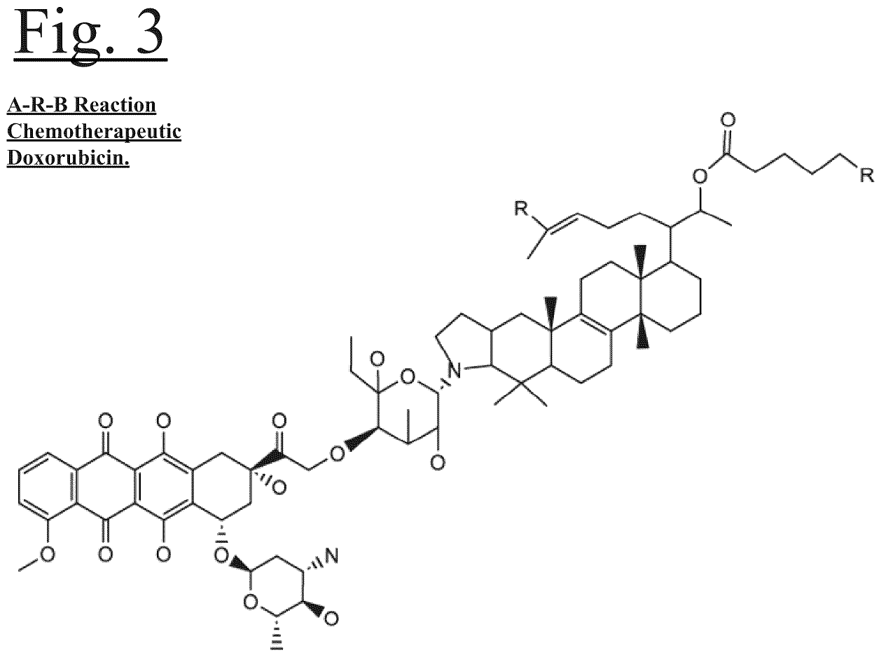

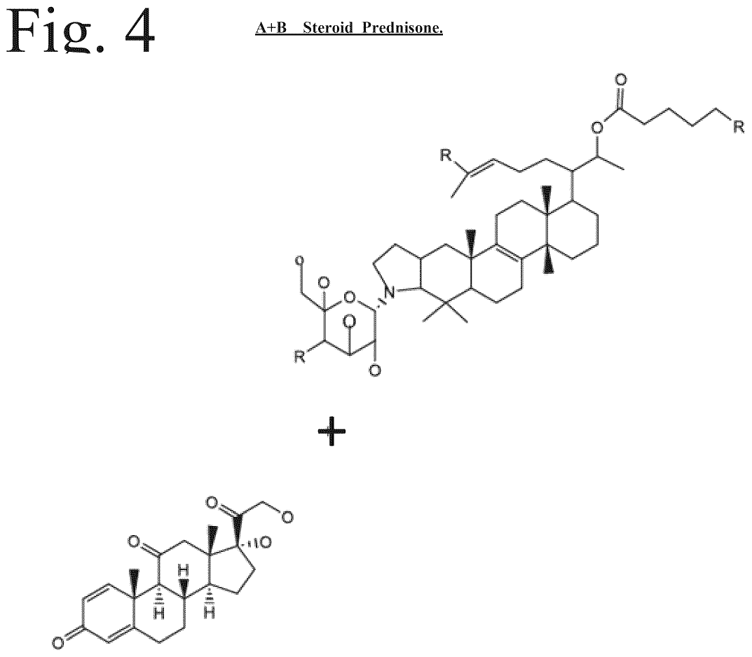

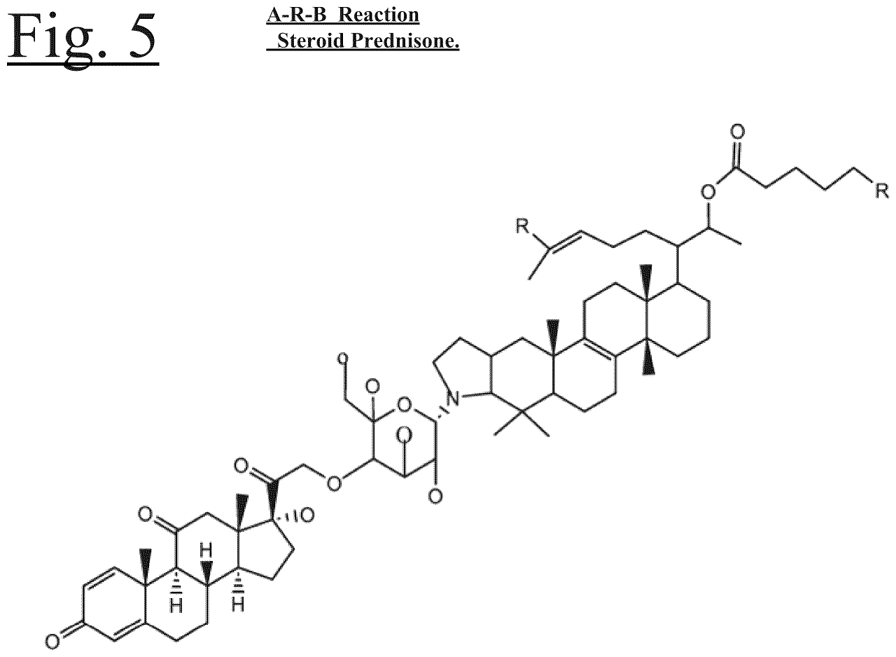

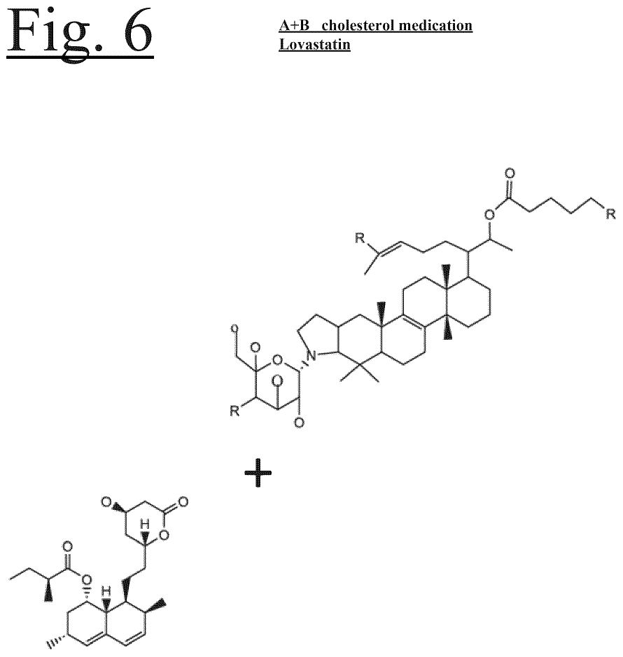

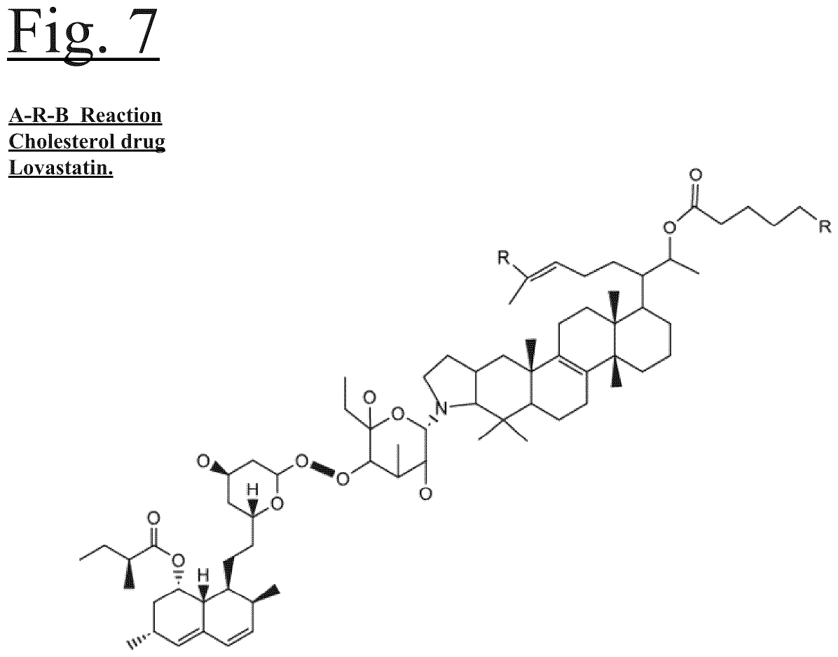

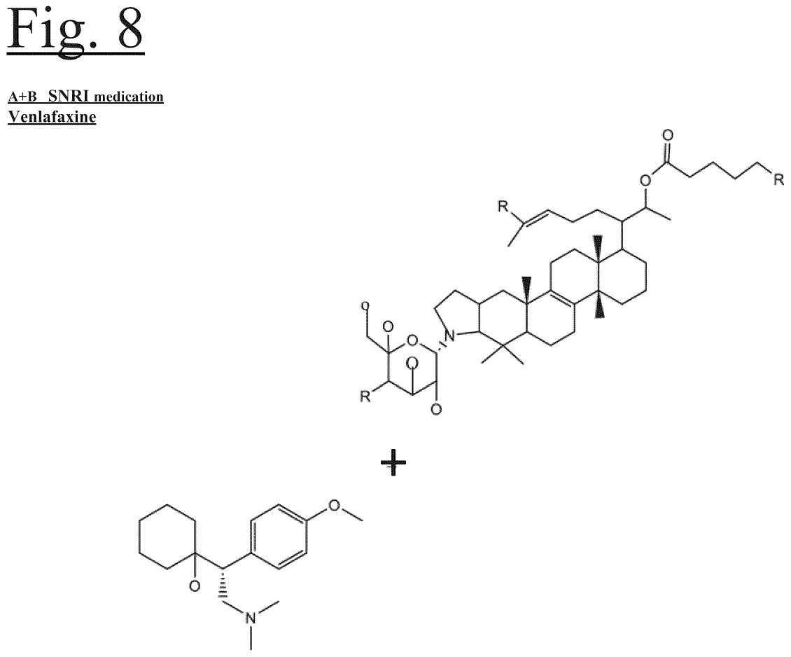

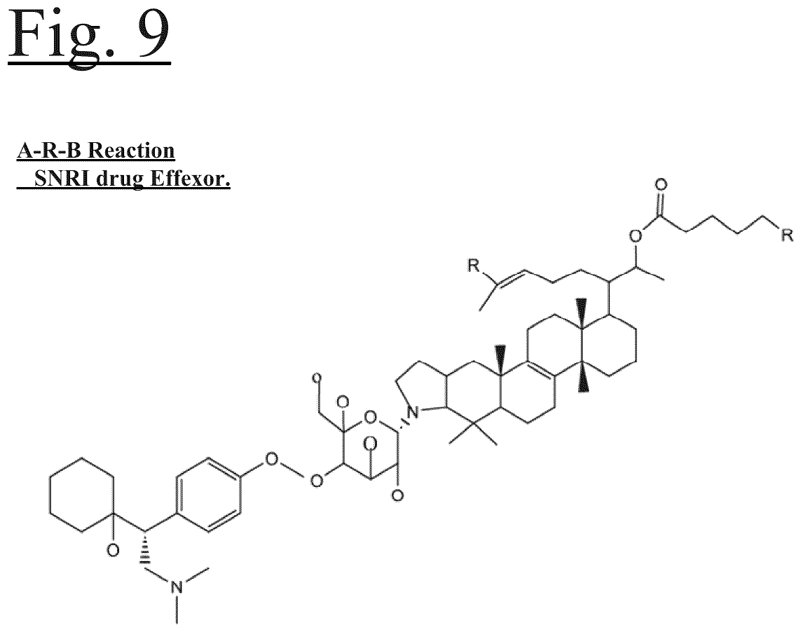

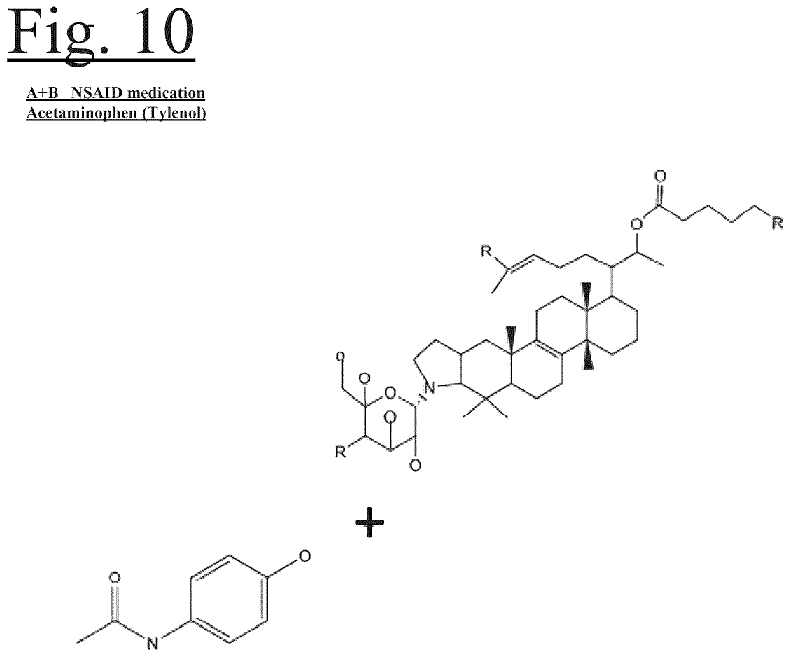

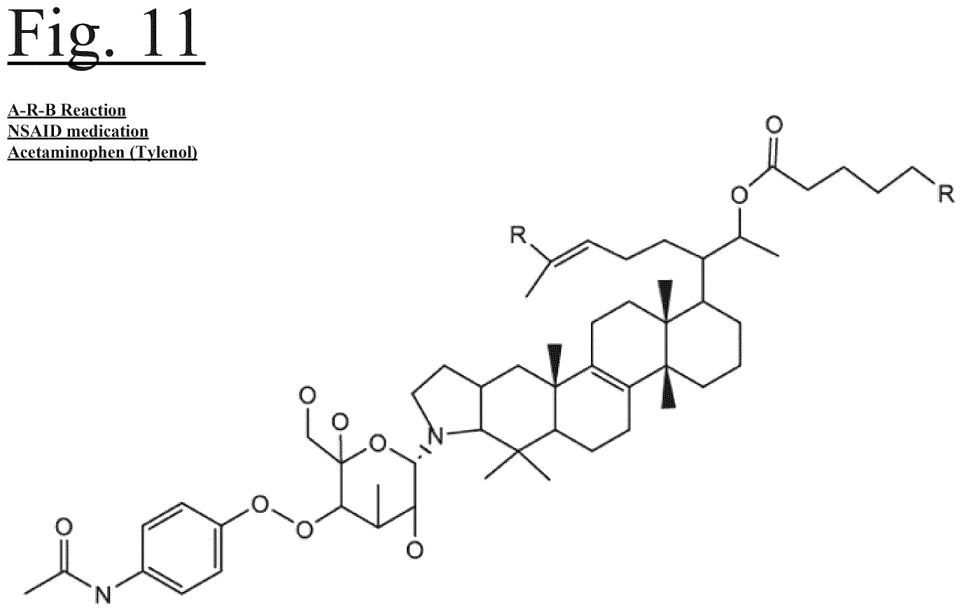

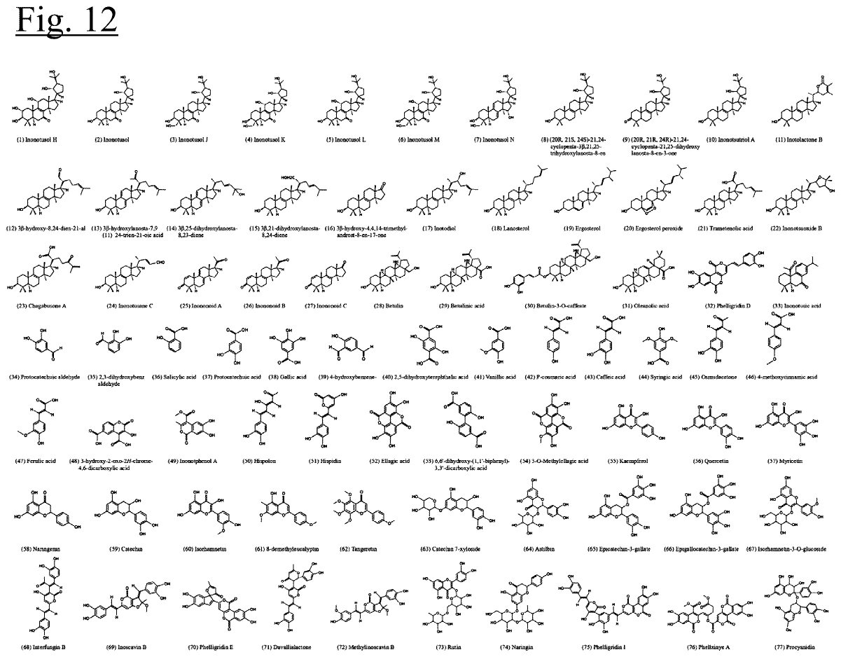

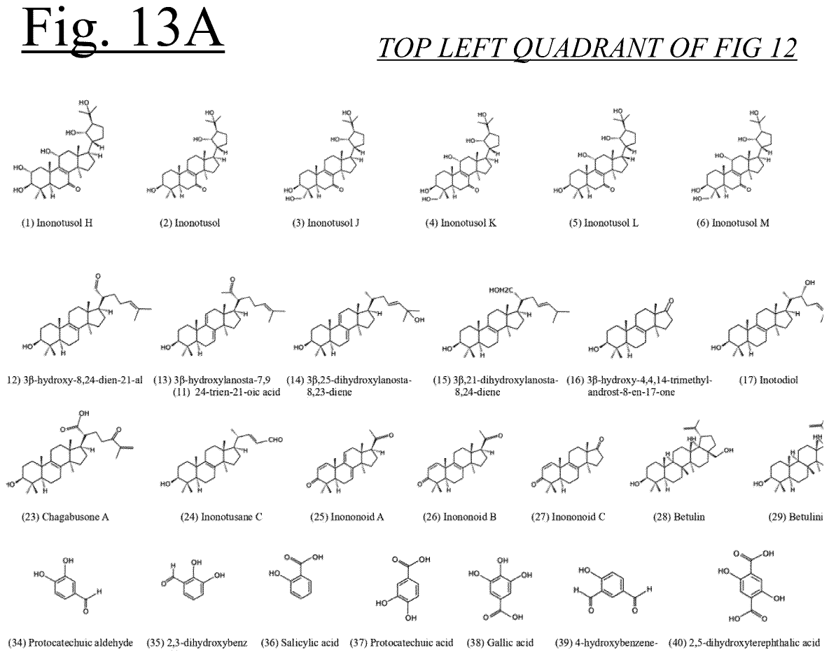

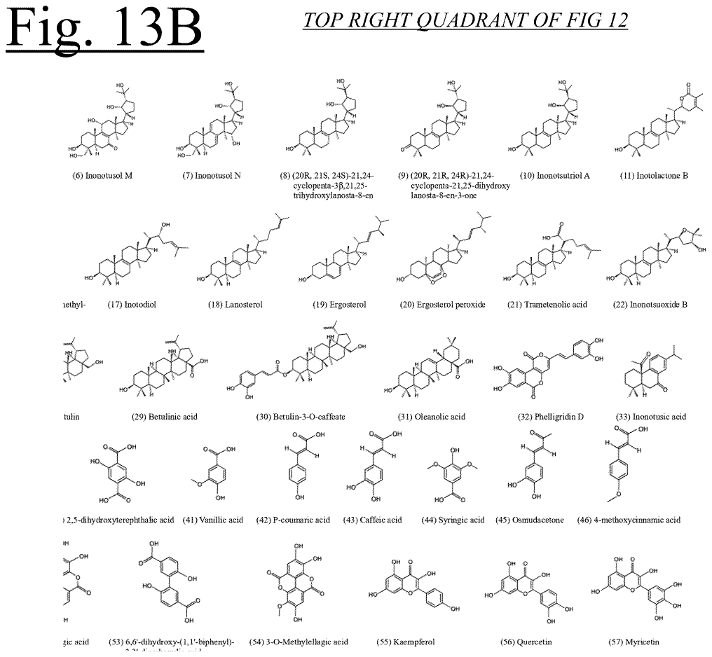

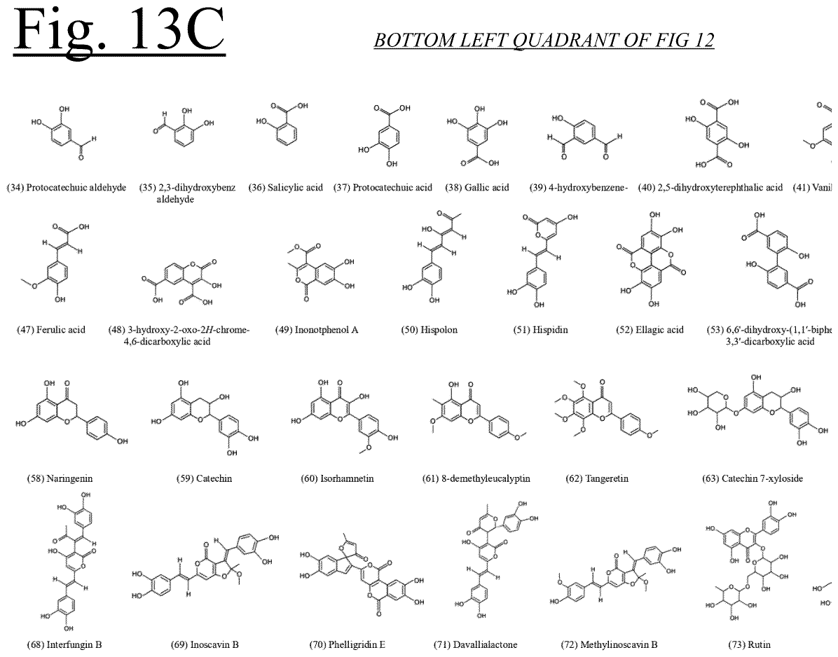

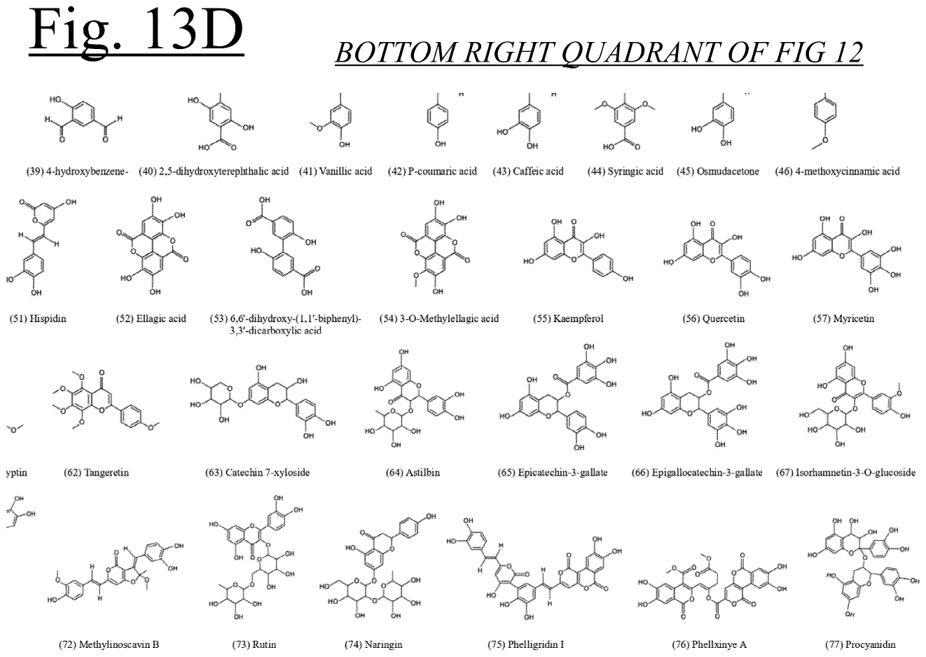

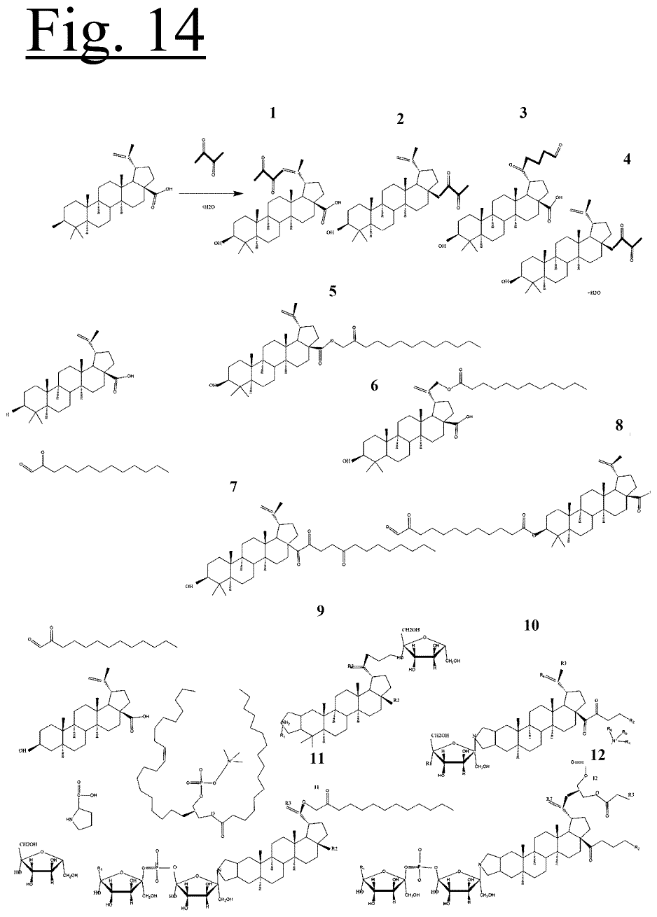

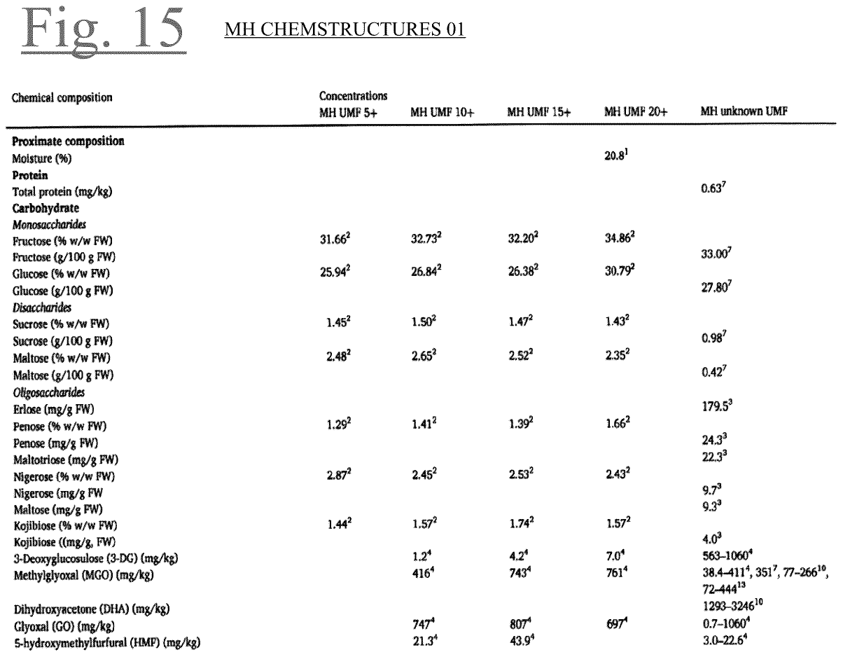

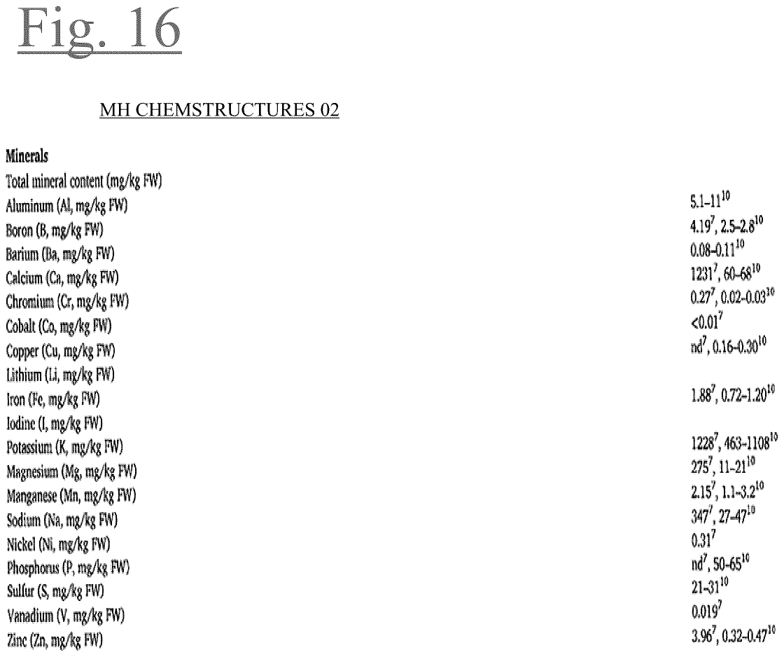

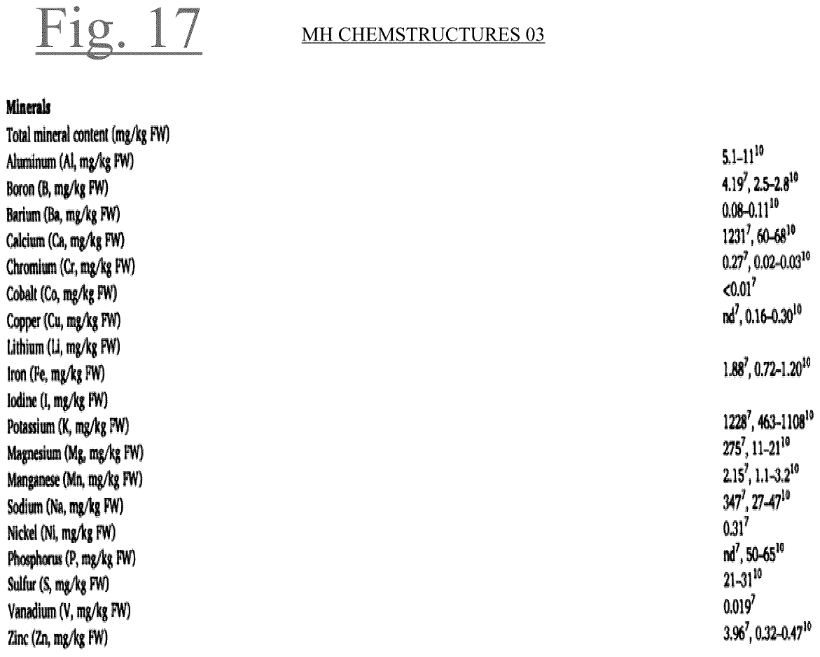

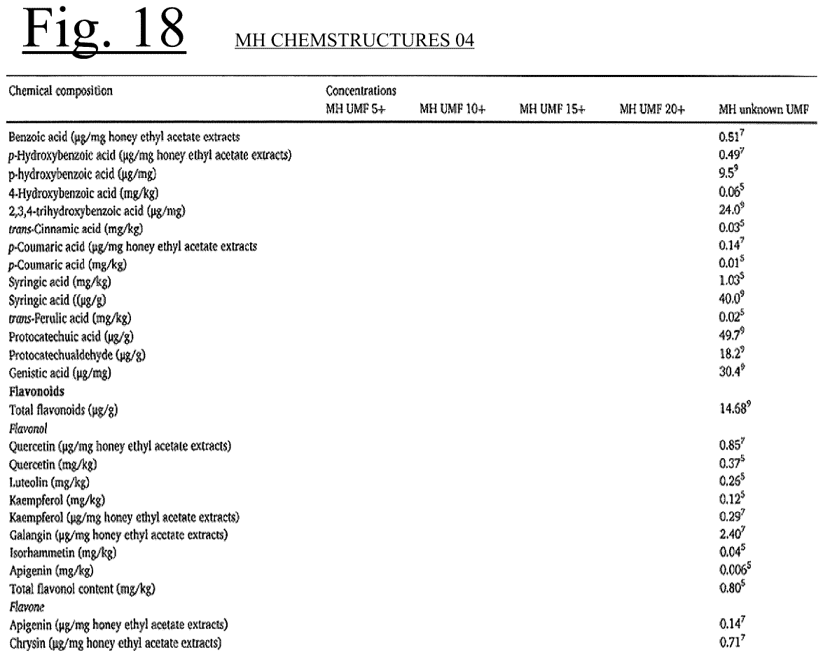

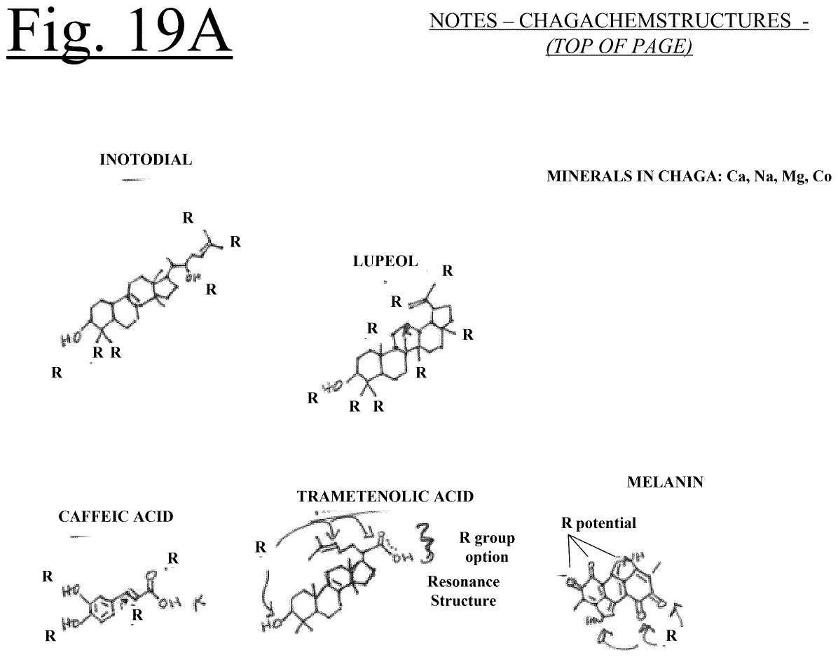

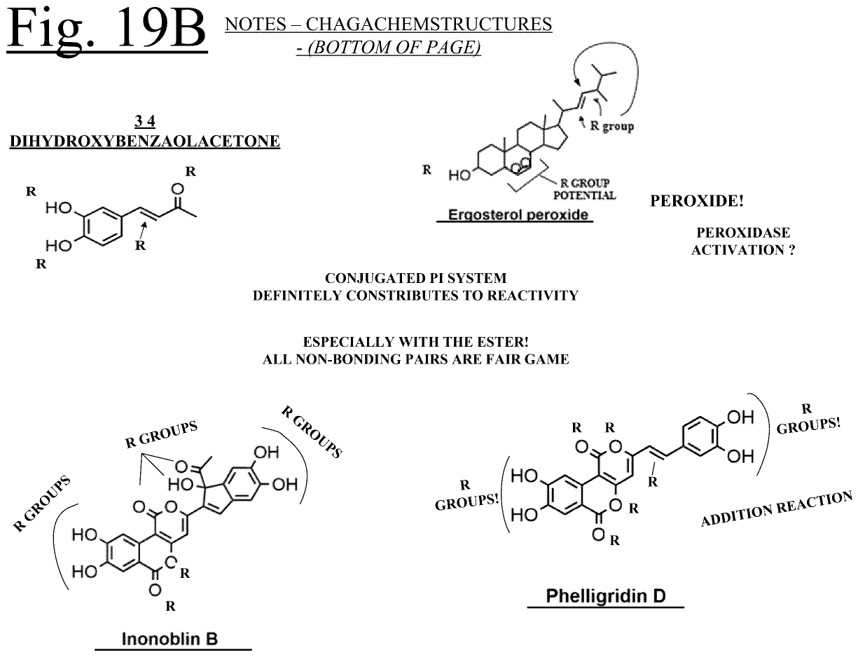

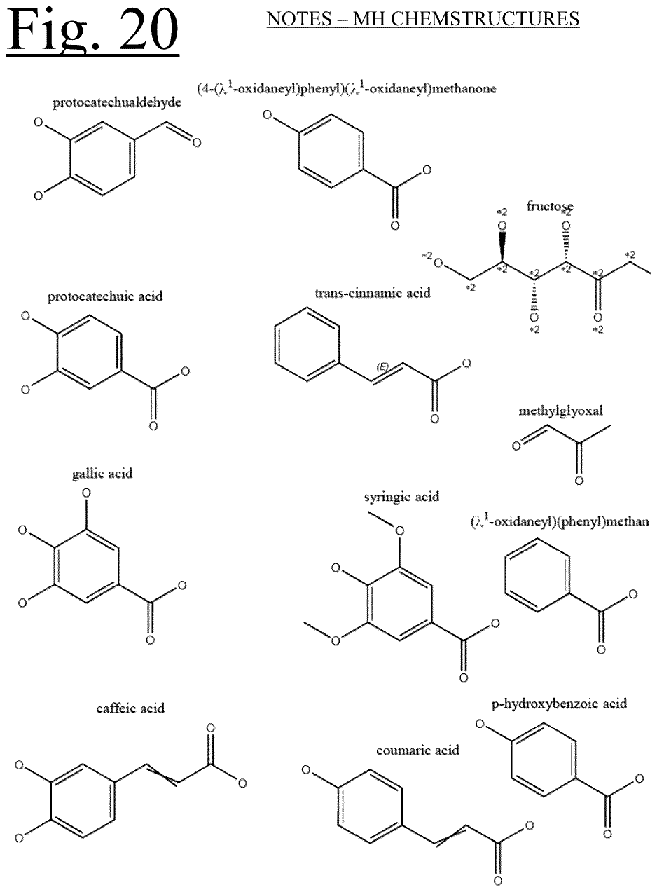

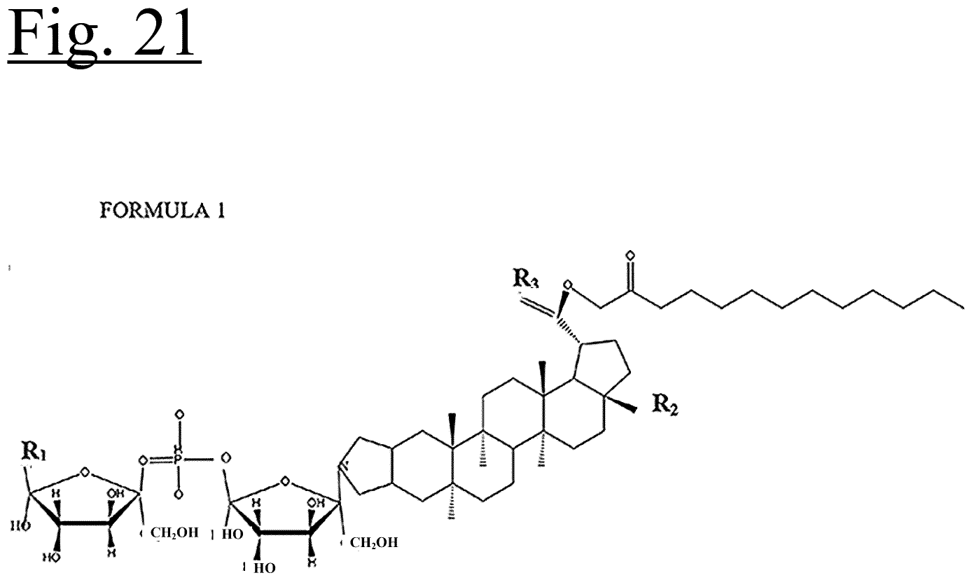

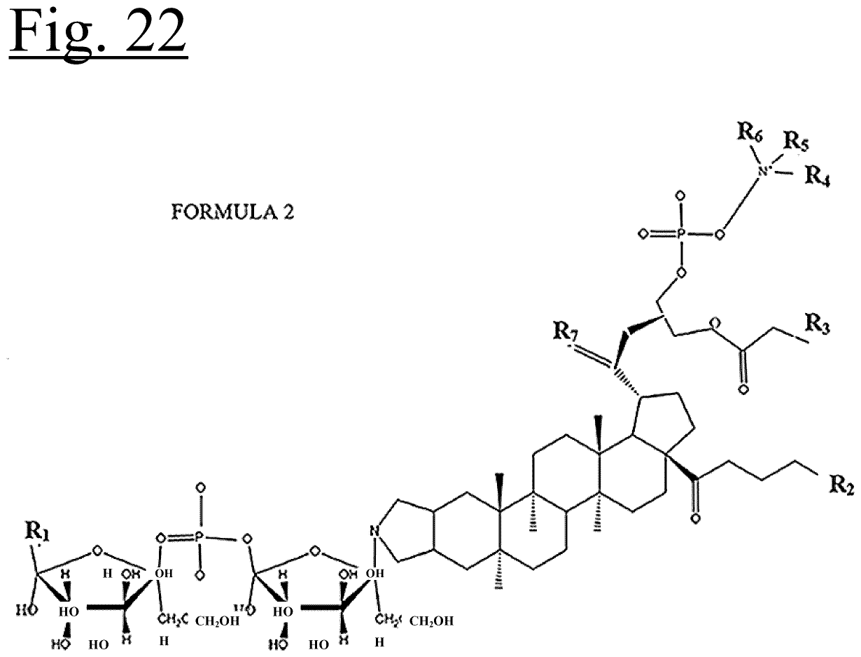

The foregoing chemical structures can be found in the published literature. They are reproduced here from the scientific publication of Ern et al. under the description, “FIG. 2. Structures of bioactive compounds isolated from Inonotus obliquus”, a published article, which is wholly incorporated by reference, said article having the following citation: Ern, P. T. Y., Quan, T. Y., Yee, F. S., & Yin, A. C. Y. (2023). Therapeutic properties of Inonotus obliquus (Chaga mushroom): A review. Mycology, 15(2), 144-161. The online citation to this article can be found at https://doi.org/10.1080/21501203.2023.2260408.

### Therapeutic Drug

The therapeutic, or pharmaceutical, drug is a human or animal drug such as a chemotherapeutic agent, analgesic, neurotransmitter, opioid, hormones or corticosteroid. The therapeutic drug benefits from the enhanced bioavailability of the triterpene in that the bioavailable triterpene enhances the bioavailability of the therapeutic drug when administered before, after or simultaneously with administration of the therapeutic drug. In particular, a therapeutic drug that causes adverse side effects when administered to a subject is given in conjunction with administration of the triterpene having enhanced bioavailability. The bioavailable triterpene increases the bioavailability of the therapeutic drug so that less drug can be administered to achieve the desired therapeutic effect. The administration of a lower dose of drug thereby reduces the onset of adverse side effects.

Additionally, the triterpene complex product having the ability to support and even increase the efficiency of mitochondrial function in all cells that contain them, including hepatocytes (liver cells) allows for much more efficient metabolism of the products from the therapeutic use of the active ingredients in the drugs that are capable of producing side effects due to bioaccumulation and/or depletion of the cellular resources needed for the cells/systems to benefit from medications without experiencing adverse events. The study described in Example 11, sets forth in the Examples section of this document, provides evidence of the increase in efficient, stabilized function of the mitochondria for ATP synthesis/signaling control of cellular metabolism and the cell life cycle.

In the embodiment directed to a method for increasing the bioavailability of a triterpene chemical compound, the Maillard reaction is utilized to initiate a reducing sugar/amine group condensation reaction. A reducing sugar is combined with an amino acid and heated at an elevated temperature under vacuum for a sufficient amount of time to form one or more Maillard reaction products.

One outcome of the Maillard reaction is referred to by those skilled in the art as “non-enzymatic browning”, a hallmark observation indicating the presence of products from the Maillard reaction. During the process of the Maillard reaction, many short-term intermediates, some known as carbocations, exist for different lengths of time. The redirection of some, if not all, of these intermediates under certain conditions described below to create a new triterpene-based product different from the standard Maillard reaction product allows for the exploitation of the existence of these temporarily vulnerable charges on the carbon-based structures without destroying the structure entirely to allow for the addition of both sugar-amine groups on one end to hold a charge such that it would allow for hydrophilic properties and fatty acids and/or surfactants through esterification partially or entirely catalyzed by the release of vaporized CO₂ and other ionic species released by and included in the Maillard reaction, to the carbon ring structure at the site of a carboxylic acid either naturally-occurring or pre-synthesized for this reaction (triterpene or other ring structures) so it is capable of hydrophobic properties at the same time. Ultimately, this creates an amphiphilic triterpene product. These properties of this new chemical entity give it the ability to interact with and pass through cellular membranes, the inner and/or inter mitochondrial membranes as well as proteins both exterior and interior to the cell simultaneously. This capacity of the product combined with the known therapeutic properties of the triterpene and/or other drugs opens the door for cells as a whole, or organelles in part, to benefit from the therapeutic compounds that would otherwise not be available. An example is the ability to transport Betulinic Acid into the mitochondria, which is known to selectively induce apoptosis in diseased cells only (not in healthy cells) by initiating the release of Cytochrome C, a member of the protein complexes responsible for oxidative phosphorylation, the redox reaction responsible for the generation of ATP from the mitochondria. This function controlled by the mitochondria is known to occur in healthy cells as a mechanism to induce normal apoptosis and the natural end of the life of a cell and is known in disease such as cancer to be prevented by the cell to evade destruction and allow for progression of the disease.

The one or more Maillard reaction products are added to a triterpene compound, and the solution is brought to a boil under vacuum, with or without a phosphate group. The triterpene is esterified with a fatty acid under similar reaction conditions to produce an emulsion or micelles. In one embodiment, the elevated temperature is approximately 100° C. (+/−15° C.). In another embodiment, the vacuum is from standard atmospheric pressure to approximately 30 psi. In another embodiment, the sufficient amount of time to form one or more Maillard reaction products is approximately 24 hours or more. In another embodiment, the triterpene is a pentacyclic triterpene, or phenolic, which is classified as a molecular structure containing at least one aromatic ring with at least one hydroxyl (—OH) group. In another embodiment, the triterpene is a pentacyclic triterpene, or phenolic, which is classified as a molecular structure containing at least one aromatic ring without a hydroxyl group, but with other functional groups such as cycloalkanes; cycloalkenes; cycloalkynes; arenes; styrenes; polycyclic aromatic hydrocarbons; heterocyclic ring structures that also contain a non-carbon element such as N, O, or S; and aromatic-amides, -amines-, -halides, -ketones, -aldehydes, -esters, -ethers, -acids, or nitro. In another embodiment, the esterification reaction is maintained under vacuum until the reaction has stopped to allow for the formation of stable products that remain intact and chemically active for a prolonged period of time.

Another embodiment is a bioavailable triterpene chemical compound. The triterpene is a reaction product produced by the method described herein for increasing the bioavailability of the triterpene by reduction, esterification and emulsification or the bioavailable triterpene is synthesized. Optionally, the bioavailable triterpene is combined with a pharmaceutically-acceptable carrier to create a pharmaceutical composition.

Another embodiment is a method of treating a disease, such as cancer, by administering one or more of the triterpene chemical compounds having enhanced bioavailability or pharmaceutical compositions containing the chemical compounds having enhanced bioavailability to a human or animal.

Another embodiment is a method for increasing the bioavailability of a therapeutic drug by combining the therapeutic drug with a triterpene having enhanced bioavailability. This can be represented with variables in the format A+B, wherein A is the bioavailable triterpene, and B is the pharmaceutical drug.

Another embodiment is a composition containing a bioavailable triterpene chemical compound in combination with a therapeutic drug. The combination is a mixture or is a reaction product in which the triterpene is joined to the therapeutic agent by an R group. In an embodiment, the R group is a saccharide (sugar molecule) or polysaccharide thereby forming a complex, which can be represented with variables in the format A-R-B, wherein A is the bioavailable triterpene, R is the saccharide (sugar molecule) or polysaccharide, and B is the pharmaceutical drug. Optionally, the bioavailable triterpene and therapeutic drug are combined with a pharmaceutically-acceptable carrier to create a pharmaceutical composition.

Another embodiment is a method of treating a disease, such as cancer, by administering to a human or animal one or more triterpene chemical compounds having enhanced bioavailability and administering one or more therapeutic drugs, sequentially or simultaneously.

Another embodiment is a method of treating a disease, such as cancer, by administering to a human or animal a pharmaceutical composition containing a mixture of one or more triterpene compounds having enhanced bioavailability in combination with one or more therapeutic drugs, in a pharmaceutically-acceptable carrier.

Another embodiment is a method of treating a disease, such as cancer, by administering to a human or animal a pharmaceutical composition containing a reaction product of one or more of the triterpene compounds having enhanced bioavailability in combination with one or more therapeutic drugs, in a pharmaceutically-acceptable carrier.

Another embodiment is a method for enhancing the bioavailability of a variety of bioactive chemicals, compounds or substances isolated from Chaga mushrooms. In accordance with the method, a reduction of Chaga mushroom is formed in a reduction solvent under pressure, an extraction of Chaga mushroom is formed in an extraction solvent, and the reduction and extraction are mixed together, or combined, in accordance with methods known to those skilled in the art to form a reduction and extraction mixture. The reduction and extraction mixture is then combined with an esterification agent. One embodiment of the esterification agent is a mixture containing proline, fructose, and a fatty acid such as a medium chain triglyceride (MCT) oil or sunflower lecithin, or other carbon chain fatty acids of various carbon chain length, with and without phospholipids, for the esterification of the reduction and extraction mixture of esterified compounds. Alternatively, sources of sugar esters capable of emulsion within otherwise non-miscible liquid compounds, or a predetermined amount of Manuka honey. The amount of esterification mixture added should be sufficient to cause esterification and facilitate emulsion at a temperature capable of inducing a reaction between the bioactive substances of the Chaga mushroom and the constituents of the esterification mixture, or Manuka honey, to form a miscible heterogenous mixture. This miscible heterogenous mixture is then combined with a medium chain triglyceride to produce a final emulsified solution containing chemical-modified compounds of the Chaga mushroom having enhanced bioavailability.

Another embodiment is a chemical compound. The chemical compound is isolated from the final solution or is synthesized and is optionally combined with a pharmaceutically-acceptable carrier to form a pharmaceutical composition.

Another embodiment is a method of treating a disease, such as cancer, by administering one or more of the chemical compounds having enhanced bioavailability or pharmaceutical compositions containing the chemical compounds having enhanced bioavailability to a human or animal.

One embodiment is a method for enhancing the bioavailability of a variety of bioactive chemicals, compounds or substances, composed of one or more triterpenes. More specifically, the triterpene is naturally occurring pentacyclic triterpenes, or a phenolic, which is classified as a molecular structure containing at least one aromatic ring with at least one hydroxyl group. Alternatively, the triterpene is a hydrocarbon ring structures without —OH groups but with other functional groups, such as, but not limited to, Cycloalkanes, Cycloalkenes, Cycloalkynes, Arenes (i.e. benzene, alkylbenzene, etc.); Styrene (benzene with a vinyl group); Polycyclic aromatic hydrocarbons (PAHs) i.e. Naphthalene, pyrene etc.; Heterocyclic ring structures that feature a ring carbon ring structure that also contain a non-carbon element such as N, O, or S; Aromatic-amides, -amines, -halides, -ketones, -aldehydes, -esters, -ethers, -acids, and -nitro.

An exemplary triterpene is betulinic acid. In accordance with the method, the triterpene is reduced in a reduction solvent under pressure in accordance with the Maillard reaction, the reduced triterpene is then combined with an esterification agent. One embodiment of the esterification agent is a mixture containing proline, fructose, and a fatty acid such as a medium chain triglyceride (MCT) oil or sunflower lecithin, or other carbon chain fatty acids of various carbon chain length, with and without phospholipids. Alternatively, sources of sugar esters capable of emulsion within otherwise non-miscible liquid compounds. The amount of esterification mixture added should be sufficient to cause esterification and facilitate emulsion at a temperature capable of inducing a reaction between the bioactive chemicals, compounds or substance and the constituents of the esterification mixture, to form a miscible heterogenous mixture. This miscible heterogenous mixture is then combined with a medium chain triglyceride to produce a final emulsified solution containing a chemical-modified compound having enhanced bioavailability.

Another embodiment is a chemical compound. The chemical compound is isolated from the final solution or is synthesized and is optionally combined with a pharmaceutically-acceptable carrier to form a pharmaceutical composition.

Another embodiment is a method of treating a disease, such as cancer, by administering one or more of the chemical compounds having enhanced bioavailability or pharmaceutical compositions containing the chemical compounds having enhanced bioavailability to a human or animal.

One embodiment of the invention is directed to a method for producing an isolate of Chaga mushroom having enhanced bioavailability. In accordance with the method, a Chaga mushroom reduction is prepared in a reduction solvent under high pressure sufficient to lower the boiling point of the constituents, a Chaga mushroom extraction is prepared in an extraction solvent, the Chaga reduction is mixed or combined with the Chaga extraction to form a Chaga reduction and extraction mixture, and then mixing or combining the Chaga reduction and extraction mixture with an esterification mixture. The esterification mixture contains proline, fructose, and a fatty acid such as medium chain triglyceride (MCT) oil or sunflower lecithin, or other carbon chain fatty acids of various carbon chain length, with and without phospholipids to cause esterification of the bioactive compounds in the Chaga reductions and extraction mixture, and may also include sources of sugar esters capable of emulsion within otherwise non-miscible liquid compounds. Alternatively, a predetermined amount of Manuka honey is combined with the Chaga reduction and extraction mixture. The amount of honey is sufficient to cause esterification and facilitate emulsion at a temperature capable of inducing a reaction between the bioactive compounds of the Chaga mushroom and the constituents of the esterification mixture or Manuka honey to form a heterogeneous mixture. This mixture is then combined with a medium chain triglyceride to form a reaction mixture.

In some embodiments the Chaga extract, and reduction mixture may be distilled before addition of the honey. The extraction/reduction mixture is distilled by heating the mixture up to a temperature where steam is first detected to come off the mixture. The matter coming off the mixture is cooled and condensed to form the distillate.

In some embodiments, the pharmaceutical composition may be administered orally. In other embodiments, particularly if the pharmaceutical composition is to be administered topically, the composition may be mixed with coconut oil or an alternative medium chain glyceride to form a gel or paste-like substance.

The type of reaction most likely induced first is known as a Maillard reaction, also called a non-enzymatic browning reaction and is responsible for the formation of intermediates that would typically form a known end-product but are redirected into a novel reaction with the bioactive compound of the Chaga mushroom and possibly other constituents found in the Chaga mushroom reductions and extracts to form novel compounds, as described herein. The Maillard reaction starts with a reaction between a reducing sugar and various types of amino acids when certain reaction parameter conditions are met. Two of the several intermediate products formed during the beginning of this reaction are known as Amadori and Heyns products. The full elucidation of a Maillard reaction remains unknown, but the various possible outcomes have been studied, as described herein, and measurements confirming the formation of these products have been achieved, as also described herein.

Another embodiment is a chemical compound having enhanced bioavailability. The chemical compound is isolated from the reaction mixture or synthesized and is optionally combined with a pharmaceutically-acceptable carrier to form a pharmaceutical composition.

Another embodiment is a method of treating cancer by administering one or more of the medicinal formulations, chemical compounds, or pharmaceutical compositions to a mammalian subject, such as a human or other mammalian animal.

### Chaga Mushroom

The compound of greatest interest that can be extracted from the Chaga mushroom is betulinic acid (also referred to herein as “BA”). Betulinic acid is a pentacyclic triterpene, recently shown to have anti-cancer properties, but lacking in any clinical results. Betulinic acid on its own has limited bioavailability, having a molar mass of 456.7 g/mol and boiling point of 550° C. Past studies of betulinic acid have pointed to potential limiting factors in its usage, including its molecular size, poor aqueous solubility and low bioavailability. In its raw organic form in the Chaga mushroom, betulinic acid is bound to chitin, the primary component of cell walls in fungi. Extraction of betulinic acid from the Chaga mushroom, therefore, must include releasing the betulinic acid from its chitin binding.

Betulinic acid has been the subject of numerous studies and has been shown to induce apoptosis and to defragment DNA by inhibiting topoisomerase in the mitochondria of cancer cells. There has also been a suggestion that betulinic acid has an ability to reverse the Warburg effect, a form of modified metabolism found in cancer cells which favor a specialized fermentation of the aerobic respiration pathway preferred by most other cells of the body. In this fermentation process, the last product of glycolysis, pyruvate, is converted into lactate or ethanol, while yielding lower amounts of ATP than in the citric acid cycle. However, it allows cancer cells to convert glucose and glutamine into biomass by avoiding catabolic oxidation into carbon dioxide, thus preserving carbon-carbon bonds, and promoting anabolism. The mechanism by which betulinic acid can interrupt the Warburg effect is currently unknown, although the induction of cellular respiration and reversal of the fermentation process of glucose, the cancer cells' main pathway of utilizing glucose, could be a meaningful adjunct process for combatting the growth of cancer cells.

In addition to these potential functions, betulinic acid along with other bioactive compounds found in the Chaga mushroom are known to break down lactate at high rates. The removal of lactate can assist with combating the acidic, immune system-hostile, environment produced by cancer cells: the relatively high quantity of lactic acid creates a more acidic environment for the cancer cells, which favor a lower pH level than healthy cells. Some literature suggests that the lactic acid may also assist in providing for cancer cells to avoid detection by the immune system, allowing them to evade destruction by the immune cells. Specific immune cells exist that, when activated, can detect and destroy cancer cells. Some of these types of cells are T-cells, specifically programmed to destroy cancer cells. When these immune cells can identify cancerous cells, they are quite capable of destroying the cancer cells.

It has also been suggested that betulinic acid may preferably create an oxidative stress load in cancer cells, and that this stress load may be a factor in how betulinic acid inhibits topoisomerase, and thus inhibiting DNA production, while seeming to be harmless to the surrounding healthy cells.

Also of interest is the high level of superoxide dismutase (SOD) in both Chaga extract and Chaga reduction. SOD's may be useful in dismutating the reactive oxygen species (ROSs) produced by the cancer cell's preferred mode of action and a part of the cellular damage cycle that induces signaling possibly linked to the spread of metastatic disease. Normal healthy cells produce their own SODs to manage oxidative stress from radical oxygen species to maintain cellular health, but cancer cells become unable to manage cellular damage and lose the ability to signal apoptosis due to cellular damage. The high content of SODs provided in the formulation may be of assistance to particular cancer cells in repairing damage and establishing a normal metabolic function once again.

There are potentially numerous bioactive compounds present in Chaga that can benefit the host's immune cell's ability to counter-act cancer progression.

### Manuka Honey

Manuka honey is a monofloral or multifloral honey produced from nectar of the manuka plant (Leptospermum scoparium), which is native to south-east Australia and New Zealand.

A test for monofloral manuka honey, adopted by the New Zealand Ministry for Primary Industries (see https://www.mpi.govt.nz/food-business/honey-bee-products-processing-requirements/manuka-honey-testing/) is that the honey has the five following characteristics:
contains 3-phenyllactic acid at a level greater than or equal to 400 mg/kg;
contains 2′-methoxyacetophenone at a level greater than or equal to 5 mg/kg;
contains 2-methoxybenzoic acid at a level greater than or equal to 1 mg/kg;
contains 4-hydroxyphenllactic acid at a level greater than or equal to 1 mg/kg; and
DNA level from manuka pollen is less than Cq36, which is approximately 3 fg/μL.

A test for multifloral Manuka honey, also adopted by the New Zealand Ministry for Primary Industries, is that the honey has the five following characteristics:
contains 3-phenyllactic acid at a level greater than or equal to 20 mg/kg but less than 400 mg/kg;
contains 2′-methoxyacetophenone at a level greater than or equal to 1 mg/kg;
contains 2-methoxybenzoic acid at a level greater than or equal to 1 ng/kg;
contains 4-hydroxyphenllactic acid at a level greater than or equal to 1 mg/kg; and
DNA level from manuka pollen is less than Cq36, which is approximately 3 fg/μL.

When the term “Manuka honey” is used herein, it refers to a honey that passes at least the DNA test (wherein the DNA level from manuka pollen is less than Cq36, which is approximately 3 fg/μL) for multifloral Manuka honey or for monofloral Manuka honey, as set forth in the previous two paragraphs.

Although not wishing to be bound by the following, several hypothetical reactions and mechanisms of action are described as follows. Manuka honey may be symbiotically contributing to the formula's function. One particularly interesting finding is that Manuka contains a high content of methylglyoxal, which is known to be produced in organisms as a side-product of several metabolic pathways, mainly glycolysis. While endogenous methylglyoxal in animals has been attributed to the formation of advanced glycation end products (or AGEs), which are used as biomarkers in aging and in the development of many degenerative diseases, research suggests that methylglyoxal derived from honey, such as Manuka, does not cause an increase in advanced glycation end products in healthy people. Furthermore, methylglyoxal in Manuka has been shown to have antibacterial activity against E. coli and S. aureus. attributed it by living cells as a sort of “self-defense” mechanism. This may give the potential for methylglyoxal to make the cancer cells more susceptible to the functions of betulinic acid, along with other compounds found in both Manuka honey and Chaga mushroom.

Manuka honey, as well as other honeys, contains high concentrations of fructose and amino acids, among other molecular compounds formed when the honey is produced. Fructose being found in high concentration, is one of a few types of reducing sugars. Many different amino acids are also found in Manuka and other types of honey in varying concentrations. The ability for fructose and amino acids to react together in an induced reaction has been shown. This type of reaction requires certain parameters and would not spontaneously occur outside of these reaction parameters. This type of documented reaction is known as a Maillard reaction and occurs when a condensation reaction is created between a reducing sugar and an amino acid. Both fructose and glucose are capable of functioning as a reducing sugar during a Maillard reaction with amino acids, undergoing a condensation reaction first to form intermediate products known as Amadori and Heyns products which are two examples of several types of intermediate products formed during a Maillard reaction. During the formation of these intermediate products, methylglyoxal is one of the reactive intermediates that assists in the formation of the final product outcomes of a typical Maillard reaction. The formation of a novel product with a fructose-amine-triterpene, or other constituent, lends to a function of interacting with cancer cells potentially through glucose receptors as well as potentially other cell signal receptors. This possible novel product likely retains a function-structure similar to that observed in other glucoside like therapeutics both found in nature and produced in pharmaceutical labs. A hypothesis proposed here is that the possible novel product could have a similar structure-function and therefore be readily consumed by the cancer cell, thus making betulinic acid and other compounds more bioavailable.

The antimicrobial properties of Manuka honey may also play a role in limiting proliferation of microbes that could be inhibitory to the immune system's destruction of the cancer cells. There is limited understanding of how certain microbes can be a catalyst in the synthesis of betulinic acid during the growing phases of wild Chaga mushroom and is also found in conversion attempts from betulin to betulinic acid in various lab work. Thus, the provision of the “raw materials” via the Chaga extract, in addition to the “blueprint” for betulinic acid with the Chaga mushroom extract, may result in the immune system's own microflora assisting or even accelerating this process of synthesizing higher content of betulinic acid and utilizing this process against cancer cells. This process can be hindered by opportunistic microbes, and interestingly some of those microbe species such as members of the Enterobacter family such as H. Pylori have been listed as a carcinogen in humans. It is postulated here that the antimicrobial functions of Manuka also help to create a friendly microbial environment that better facilitates the synthesis and reaction of betulinic acid and other compounds. This function may also facilitate the increasing bioactivity of betulinic acid, and allow for better utilization of betulinic acid and other compounds by healthy immune cells.

Another property of Manuka honey is its potent anti-biofilm properties, which may be important due to the nature of bio-film creation by hostile microbes. One of the many, not fully understood, actions against bio-films is to reverse genetic mutations in microbes that have become resistant to antimicrobial treatments, which makes them more easily eradicated by Manuka's own antimicrobial function. Bee defensin is prevalent in all honeys, but the Manuka honey displays some unique protective functions that are not peroxidase based. This may be important in that the Manuka honey compounds do not destroy friendly microbes that could be utilized in synthesis actions as well as protecting healthy cells.

Manuka honey has a relatively low pH, about 3.5-4.5, which contrasts with the higher pH of the Chaga extract and reduction. Furthermore, the lower pH of the honey may contribute to the formula's action on cancer cells because it may be less affected by one of the cancer cell's primary defense mechanisms, i.e., the reduction of pH levels to evade immune cell attacks. This also may permit Manuka to inhibit microbial growth, stimulate the bactericidal actions of macrophages in the host's immune system and, in chronic wounds, to reduce protease activity and increase fibroblast activity and oxygenation.

In addition to the earlier functions discussed, recent studies have demonstrated that Manuka honey can exert anti-proliferative effects against cancer cells. These anticancer properties can involve different processes, including inducing apoptosis in cancer cells through the depolarization of the mitochondrial membrane, inhibiting cyclooxygenase-2 by various constituents (like flavonoids), releasing cytotoxic H2O2, and scavenging of reactive oxygen species (or ROSs). The main mechanism by which Manuka exerts its anti-proliferative effect is through the activation of mitochondrial apoptotic pathways, involving the stimulation of the initiator, caspase-9, which determines the activation of the executioner, caspase-3. This last function is also shared by Chaga mushroom. Moreover, it can induce apoptosis via the activation of PARP, the induction of DNA fragmentation and the loss of Bcl-2 expression.

In vivo, Manuka honey has been shown to be effective in decreasing tumor volume and supporting apoptosis of tumor cells in a mouse melanoma model, reducing colonic inflammation in inflammatory bowel disease in rats, restoring lipid peroxidation and improving antioxidant parameters. Studies show Manuka honey has no detrimental effect in relation to advanced glycation end products nor to a change in gut microbiota homeostasis. This is important as it relates to the unknown properties of how methylglyoxal relates to the progression of certain chronic diseases.

### Carrier

Coconut oil or an alternative medium chain triglyceride may be used to carry the formulation and contribute biochemically to its function, particularly where the formulation is applied topically. The medium chain triglyceride adds a protective layer when the formulation is applied to an open skin lesion as well as subdermal tumors. The carrier is preferably a soft solid, that can be applied as a paste, or a liquid, and can preferably dissolve the Chaga extraction/reduction as well as the honey. In some embodiments the carrier/formulation mixture may be sprayed on to the area to be treated. Coconut oil is a white, solid fat that melts at a temperature of approximately 25° C. to make a clear thin liquid oil. Alternative medium chain triglycerides, for example palm oil, may be liquid at room temperature.

### Preparation of Bioactive Compounds Having Enhanced Bioavailability

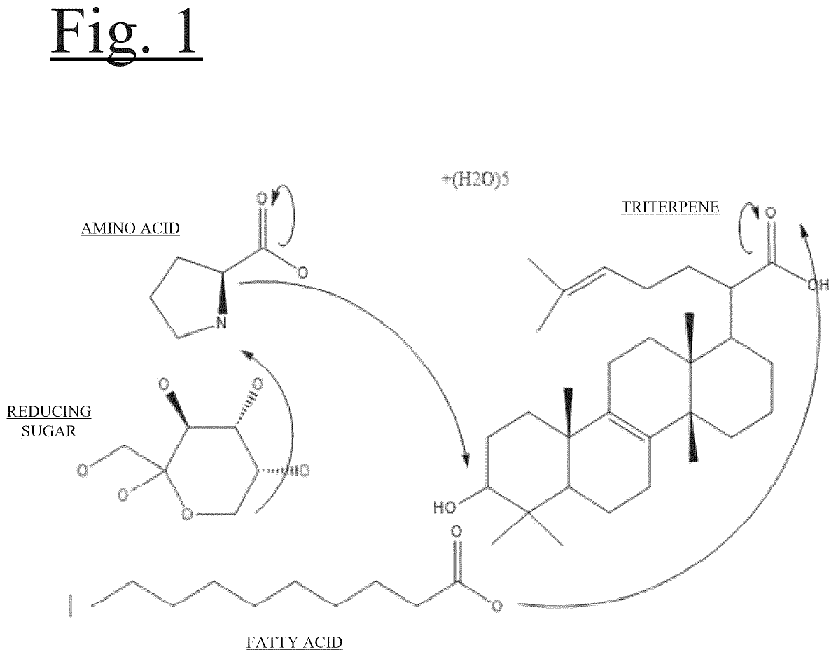

Numerous bioactive compounds can be isolated from Chaga mushrooms. The chemical structures of most of these compounds is provided in FIG. 1. However, these compounds exhibit poor bioavailability when administered to mammalian patients. The methods described herein utilize an unpredicted reaction mechanism to create reactive sites on these bioactive compounds that enhance bioavailability. Applicant unexpectedly discovered the ability to manipulate organic compounds, known and unknown, that possess the potential for reaction sites to be created by altering steric hindrance, utilizing resonance alterations, or creating electronegative differences through the reactions described herein. In this way, the underlying, typically stable, bonds could be altered or changed where there may or may not already be sites of molecular interaction or reactivity.

A detailed description of the reaction process utilized to create reaction sites on the bioactive Chaga compounds is provided below.

### Preparation of a Chaga Extraction

An extraction of Chaga mushrooms is using the following approach. The Chaga mushroom is chopped to increase the surface area exposed to liquid. In some embodiments, the Chaga is chopped so that 90% of the granules have a maximum dimension of less than 7 mm in maximum dimension, preferably less than about 5 mm, more preferably less than about 3 mm and more preferably less than about 2 mm.

The chopped Chaga is then mixed with an extraction solvent. The extraction solvent typically includes a nonaqueous solvent and may be a solvent typically used for food extraction. Examples of nonaqueous solvents include a short chain alcohol such as ethanol, a short chain glycol such as propylene glycol, a short chain acid, such as methanoic acid, ethanoic acid or lactic acid, a short chain ketone such as acetone, or a short chain ester such as ethyl acetate or n-butyl acetate. If a solvent is used that would be toxic to the patient, it can be removed using standard chemical processing means. The extraction solvent is preferably present in an amount such that the dry volume of chopped Chaga is about one half the volume of the extraction solvent, although more or less may be used.

Water may be included in the extraction solvent, with the ratio of non-aqueous solvent to water in the extraction solvent being between about 5:95 to 100:0. Preferably the ratio of non-aqueous solvent to water is in the range 95:5 to 60:40, more preferably in the range 90:10 to 70:30. In other words, the Chaga is mixed into a liquid that includes at least one non-aqueous solvent. The extraction solvent dissolves components of the Chaga released from the mushroom into the solvent. The non-aqueous solvent tends to dissolve the more lipophilic components of the Chaga while the water tends to dissolve the less lipophilic components of the Chaga, because the water is commonly more polar than the non-aqueous solvent. It will be appreciated by those skilled in the art that other non-aqueous solvents may be used.

The dielectric constant, ε, of the extraction solvent is preferably less than 50, more preferably less than 40 and may even be less than 30. For example, where the liquid solvent is 100% ethanol, the dielectric constant, v ethanol, is approximately 24.5. In a mixture of miscible liquids, the dielectric constant may be calculated by taking a volumetric average of the dielectric constants. For example, in a liquid containing 90% ethanol by volume and 10% water (ε water=80.1) by volume, the dielectric constant of the mixture is given by (0.9× ε ethanol)+(0.1×ε water)=30.2. The dielectric constant of the liquid is an indicator of the polarity of the solvent mixture.

Larger values indicate that the solvent polarity is higher, which may result in the extraction of less lipophilic constituents of the Chaga mushroom. Lower values of dielectric constant indicate that the solvent polarity is lower, in which case the solvent may be more effective at extracting more lipophilic components from the Chaga mushroom. The solvents used may be either protic or nonprotic. A mixture of ethanol and water is a mixture of two protic solvents.

The Chaga/liquid mixture is then left for a predetermined time suitable for components of the mushroom to be extracted. In some embodiments, the mixture is left at room temperature for more than 48 hours, more than a week, preferably more than a month, more preferably more than two months and even more preferably more than three months. The mixture may be covered or sealed to prevent evaporation of the liquid components. The extraction time may be shorter if the mixture is held at an elevated temperature, for example 90° F., 100° F., or higher, for example up to about 160° F. and/or if the mixture is agitated, for example using a magnetic stirrer. The temperature of the mixture is kept below the boiling point of the liquid.

Another approach to forming an extraction is to use an ultrasonic extraction method, in which ultrasound waves are introduced to the Chaga/liquid mixture. Ultrasonic waves, which may be generated by an ultrasonic probe or other suitable ultrasonic generator travel through the liquid creating alternating high-pressure/low-pressure areas, which can result in acoustic cavitation. This, in turn, can result in locally extreme temperatures and pressures, heating/cooling rates, pressure differentials and high shear forces. When cavitation bubbles implode on the surfaces of the Chaga parts, mass transfer from the Chaga parts into the liquid is enhanced. Under ultrasonic extraction, the Chaga/liquid mixture is exposed to ultrasonic waves for a predetermined period of time, typically a few minutes to hours, to transfer components from the Chaga into the liquid. Ultrasonic extraction may take place at room temperature or at elevated temperatures.

The mixture is then filtered to remove the solids. Any suitable method of filtering may be used, depending on how the liquid extraction is to be used. For example, if the liquid extraction is to be taken orally or applied topically, then the presence of some small Chaga particles, typically <1 mm, may be acceptable, and a filter as coarse as a tea strainer may be acceptable. If, on the other hand, the extract is to be sprayed onto the recipient, then particles as large as 1 mm may block the spray equipment and a finer method of filtering, for example using a filter paper may be used. The extraction is preferably stored in an acid-resistant container, for example a glass container.

The Chaga extract typically has a pH level ranging from 4-9. Generally, the pH of the Chaga extract is lower with longer extraction times and with less polar liquids.

### Method of Preparing a Chaga Reduction

A reduction of Chaga mushrooms is made by immersing the chopped Chaga in water, at an elevated temperature for a period of time, or cycled through elevated temperatures for a number of times. In one approach, the Chaga parts are exposed to a boiling or greatly elevated temperature. The Chaga mushroom is chopped so that 90% of the granules have a maximum dimension of less than about 75 mm in maximum dimension, preferably less than about 50 mm, more preferably less than about 30 mm and more preferably less than about 20 mm. The Chaga may also be chopped as finely as discussed above with respect to the extraction, e.g., so that 90% of the granules have a maximum dimension of less than 7 mm in maximum dimension, less than about 5 mm, less than about 3 mm and even less than about 2 mm. The Chaga reduction is made using a solvent that contains at least water and, optionally another liquid, although the solvent used in a reduction is more polar than that used in an extraction.

In one approach, the chopped Chaga is placed in a reduction solvent such as water, preferably in a volume ratio of about 1:40 to about 1:2. Preferably the chopped Chaga is covered by the reduction solvent. The reduction solvent may be brought to a boil and then allowed to cool down again in a cycle. During the boiling/cooling cycle, the reduction solvent is brought to a strong boil for a time and kept at an elevated temperature for a time before being allowed to cool off. In some embodiments, the reduction solvent is brought to a strong boil for 1-10 minutes, more preferably 4-5 minutes, and then brought to a simmering boil for about 10-60 minutes, preferably 15-45 minutes, more preferably 20-30 minutes, with the remainder of the cycle permitting the water to cool. An exemplary cycle includes heating to a boil, achieving a rolling boiling for about 4-5 minutes, reducing to a simmering boil for about 20-30 minutes, and cooling for the rest of the cycle. The cycle may take, for example, one hour or more.

In another approach to forming a reduction, the chopped Chaga is heated to an elevated, but not boiling, temperature, for example 160° F. or higher, for a period of time, for example 24-96 hours, 3-7 days, or even longer under a pressure of 5-15 psi+/−4 psi.

The reduction solvent may include a mixture of different liquid solvents. The dielectric constant, ε, of the reduction solvent is preferably more than 50, preferably more than 60 and may even be more than 70. For example, where the liquid solvent is 100% water, the dielectric constant, ε water, is approximately 80.1. Here, values of dielectric constant are provided as the d.c. (dielectric constant). In a mixture of miscible liquids, the dielectric constant may be calculated by taking a volumetric average of the dielectric constants. For example, in a liquid containing 90% water by volume and 10% ethanol by volume, the dielectric constant of the mixture is given by (0.9×ε water)+(0.1×ε ethanol) 74.5. The dielectric constant of the reduction solvent is greater than the dielectric constant of the extraction solvent, for example, by more than 10, more than 20, or even by more than 30. In an example where an extraction solvent is 9:1 parts ethanol to water by volume, the dielectric constant is about 30.2, whereas in in a reduction solvent containing 9:1 parts water to ethanol by volume, the dielectric constant is about 74.5, a difference of about 34.

The mixture is then filtered to remove the solids. Filtering may be performed after allowing the water to cool. Any suitable method of filtering may be used, depending on how the liquid reduction is to be used. For example, if the liquid reduction is to betaken orally or applied topically, then the presence of some small particles, typically <1 mm, may be acceptable. If, on the other hand, the reduction is to be sprayed onto the recipient, then particles as large as 1 mm may block the spray equipment and a finer method of filtering, for example using filter paper, may be used.

In some approaches, the reduction is formed using a mixture of water and some other, nonaqueous, solvent, such as a food grade solvent. For example a nonaqueous solvent may be a short chain alcohol such as ethanol, a short chain glycol such as propylene glycol, a short chain acid, such as methanoic acid, ethanoic acid or lactic acid, a short chain ketone such as acetone, or a short chain ester such as ethyl acetate or n-butyl acetate If a solvent is used that would be toxic to the patient, it can be removed using standard chemical processing means.

### Method of Preparing an Extraction/Reduction Mixture

The extraction may be mixed with the reduction to produce an extraction/reduction mixture (ER mixture). The ratios of volumes of the extraction and reduction used to form the ER mixture may cover a wide range, for ex<:1 mple from 50:1 (i.e. 10 parts extraction to 1 part reduction) to 1:50 (i.e. 1 part extraction to 50 parts reduction), preferably from 5:1 to 1:20, more preferably 1:1 to 1:10 and even more preferably 1:3 to 1:7. The volume ratio of extraction and reduction in ER mixture may be around 1:5. In some approaches, the extraction is added to the reduction in an acid-resistant container, for example a glass container.

### Proposed Reaction Mechanism

Although not wishing to be bound by the following proposed reaction mechanism theory, the Maillard reaction occurring in the method of preparing the Extraction/Reduction mixture described above most likely induces a condensation reaction. This might start with fructose and proline, an amino acid in high concentration in honey, and the product of that reaction reacts with methylglyoxal/betulinic acid, since methylglyoxal is a precursor to protein glycation. The condensation reaction with betulinic acid/methylglyoxal and then one or more of the fatty acids could also be set in motion during certain phases of the Maillard reaction when some of the intermediates are very reactive. If this is happening, it could be creating a glycoside-like molecule from the fructose/proline/methylglyoxal/betulinic acid with a fatty acid component. If possible, this could help explain the non-polar/polar indications found in the formula as some of the pH tests show various pH levels in the final assembly. This could also explain the fluorescent nature of the formula found in the first in vitro studies, the final measurements had to be done using an alternative to the fluorescence measurements because of absorbance. If the resulting active ingredient has a glycoside quality, and it survived the digestive system intact, it could be attractive to the glucose receptors on cancer cells and promote binding.

Additional theories for this reaction mechanism are that the Maillard reaction is initiated at the temperature range in which the above-described reaction mixture is incubated; the Maillard reaction has the capacity to induce a condensation reaction, especially if fructose is involved, as it reacts with available carbonyl groups; a reaction between fructose and proline is highly likely, and the product of that reaction is commercially available from Toronto Research Chemicals; the reaction could also involve glycine, or other amino acids; the Maillard reaction could protect the molecule from breaking down in the stomach and small intestine, making it possible to reach the cancer cells while intact; the combination of polar and non-polar starting materials reacting might facilitate a spherical structure, a structure that has been thought to be observed in some of lab tests of substances provided herein; if the structure of the molecule created is a micelle-like structure, that could explain the hydrophobic/hydrophilic characteristics observed; a micelle structure could facilitate transport through the bloodstream in or around the small intestine and is feasible given the observed particle size, it seems feasible.

### Method of Producing a Medicinal Formulation

Formulations may be formed using any combination of the products listed above, including the Chaga extraction, the Chaga reduction, the distillate, and the remaining fraction. In addition, the formulation may include a honey such as a manuka honey, and a carrier, such as coconut oil or other medium chain triglyceride. For example, an oral formula may include the Chaga extraction, the Chaga reduction and the Chaga distillate in a suitable ratio. Typically, the reduction is present in an oral formula at a greater amount than the Chaga extract or the distillate, although this is not a necessary condition. For example, in the formulation, the ratio of reduction to extract may lie in the range of 100:1 to 1:100, preferably in the range 20:1 to 1:10 and more preferably in the range 20:1 to 1:1. The ratio of reduction:remaining fraction may also lie in the range of 100:1 to 1:100, preferably in the range 20:1 to 1:10 and more preferably in the range 20:1 to 1:1. The ratio of the extract:remaining fraction may lie in the range of 100:1 to 1:100, preferably in the range 1 0:1 to 1:10 and more preferably in the range 5:1 to 1:5. In one particular example, the weight ratio of the Chaga reduction, Chaga extract and distillate is 5:1:1. Other ratios may be used within the range limits discussed.

Additionally, if administered orally, the formulation may also include manuka honey typically, but not necessarily, in an amount less than the amount of distillate or extract. For example, the weight ratio of Chaga reduction, Chaga extract, distillate and manuka honey in a formulation may be 10:2:2:1. Other ratios may be used. In particular, the weight ratio of reduction:honey may lie in the range 10:0 to 10:10, or in the range 10:0.1 to 10:10, although ratios higher than about 10:2 may change the consistency of the formulation and also become expensive.

The formulation may also include a carrier, such as coconut oil or other medium chain triglyceride typically, but not necessarily, in an amount less than that of the distillate. The weight ratio of reduction:carrier may lie in the range 10:0 to 10:50, or 10:0.1 to 1:50, depending on the method in which the formulation is to be administered. For example, in a liquid formulation to be applied to a horse's feed, the weight ratio of reduction:carrier may be 1 0:0 to about 10:2. In one example of a liquid formulation, the weight ratio of Chaga reduction, Chaga extract, distillate and coconut oil in the formulation may be 10:2:2:1. Higher amounts of carrier may be used for a more solid formulation, such as a salve. In an example, the reduction:carrier weight ratio may be 10:10. Other ratios may be used.

The formulation may include both manuka honey and a carrier such as coconut oil or other medium chain triglyceride. The amount of each of the manuka honey and the carrier may be, but is not required to be, less than that of the extract and distillate. For example, the weight ratio of Chaga reduction, Chaga extract, distillate, manuka honey, and coconut oil in a formulation may be 10:2:2:1:1. Other ratios may be used.

An alternative formulation is provided, which is a reaction between the Chaga extract described above with Proline, Fructose and a fatty acid such as medium chain triglyceride oil or Sunflower lecithin, or other surfactant, for the esterification process of betulinic acid with fructose and proline to create a fructose-amine-triterpene. These are the isolated starting materials originally supplied by the Manuka Honey. The fully synthesized product is suspended in the original formulation, and all material is food grade and FDA approved for sale in original form.

### Administration of Medicinal Formulation

The medicinal formulation may be administered in any suitable manner known to those skilled in the art. The medicinal formulation is a liquid and may be administered or applied to solid food. For example, the formulation may be orally consumed by the patient or with the formulation may contain additives, such as sugar, salt, etc., that may be used to alter the flavor to a taste preferred by the patient. In other approaches, the formulation may be added to another liquid to be drunk by the patient. For example, in the case of a human patient, the formulation may be added to a drink such as coffee, tea, a carbonated soda or the like. In the case of an animal patient, such as an equine patient, the medicinal formulation may be added to the animal's water. In other approaches, the formulation may be mixed together with food. For example, in the case of a human patient, the formulation may be added to soup, included in gravy served over meat, vegetables or potatoes, or included in a sauce served with, e.g. pasta or meat. In the case of an animal patient, such as an equine patient, the formulation may be fed directly or may be sprinkled over the horse's feed. When fed directly, an additive may be included to mask the taste of the formulation. One example of a taste-masking agent is sugar. The formulation is well absorbed in pelleted feed. Whole grain feeds and hay, on the other hand do not absorb the formulation as well, in which case the formulation may include a coating agent such as medium chain triglyceride oil so that the formulation adheres to the feed.

In other approaches, the formulation may be applied topically. In some cases, the formulation may be made more viscous when applied topically, for example by adding a more viscous carrier such as coconut or other medium chain triglyceride oil, or a thicker oil. The formulation may be applied topically using a towel, piece of cloth, or pad soaked in the formulation. Another approach is to apply a pad, poultice or the like, that has been soaked in the formulation, to the area to be treated and to hold the pad, poultice or the like in place against the skin. The pad, poultice or the like may be held in place using, for example, adhesive strips, a bandage or any other suitable method. Where the formulation includes a relatively large fraction of carrier, the formulation may have the consistency of a salve that can be spread on the area of concern.

In other approaches, the formulation may be included in a gel, paste or lotion that may be applied to the area to be treated.

In other approaches, the formulation may be sprayed on the area to be treated. In such a case, it may be preferred to include the formulation with a viscous carrier, and for the formulation to be more finely filtered than needed for oral administration, in order to prevent clogging the spraying equipment.

Additional methods of administering the medicinal formulation are provided below with respect to administration of the pharmaceutical compositions containing the isolated or synthesized chemical compound represented by Formula 1 and Formula 2.

The medicinal formulation may be administered to a patient at suitable dose levels. For example, an oral formulation may be dosed daily at between 0.1 ounce per 1000 lb. (approximately 0.065 mL/kg) of patient weight and 1 ounce per I lb. (approximately 6.5 mL/kg) of patient weight. In some embodiments, the medicinal formulation is administered at a daily dosage of 1 ounce per 100 lb. of patient weight (approximately 0.65 mL/kg). The medicinal formulation may be provided in a single daily dose or in two or more smaller doses in a day.

The medicinal formulation may be administered with other therapies. For example, a patient taking the medicinal formulation as described herein may also be on a standard antibiotic regimen.

### Chemical Compounds and Methods of Production

Novel bioactive chemical compounds having enhanced bioavailability are provided herein. The novel compounds are isolated from the end product of the reaction mixture or are synthesized. The reaction sites of the bioactive chemical compounds are located on opposite ends of the chemical compound so that the compound has a hydrophilic end and a hydrophobic end. This artificially-created polarity of the compound is believed to give the compound the enhanced bioavailability observed. All reaction sites have the potential to be converted to “R” groups using the functional groups listed below.

In one embodiment, the chemical compound having enhanced bioavailability is represented by one of the chemical formulas in FIG. 1, wherein one or more of the reaction sites are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester/ester quat, carboxylic acid (COOH), acid anhydride/acetic anhydride, acyl halide, or methyl.

In another embodiment, the chemical compound having enhanced bioavailability is represented by one of the chemical formulas in FIG. 1, wherein R₁-R₃ are hydroxyl.

In another embodiment, the chemical compound having enhanced bioavailability is represented by one of the chemical formulas in FIG. 1, one or more of the reaction sites are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester/ester quat, carboxylic acid (COOH), acid anhydride/acetic anhydride, acyl halide, or methyl.

In another embodiment, the chemical compound having enhanced bioavailability is represented by one of the chemical formulas in FIG. 1, one or more of the reaction sites are, independently, hydroxyl.

The chemical compounds provided above are isolated from the medicinal formulation as described in more detail in the Examples.

Confirmation of the isolate or synthetic compound as a derivative of one of the compounds set forth in FIG. 1 having enhanced bioavailability is determined by analytical techniques such as HPLC, GC, Mass Spectrometry or NMR.

The chemical compounds are synthesized using the reaction pathways shown in FIG. 58. A preferred reaction temperature for the synthesis is from 240 to 260° C.

### Pharmaceutical Compositions and Methods of Production

The pharmaceutical composition provided herein contains one or more of the chemical compounds described above in combination with a suitable carrier. The pharmaceutical composition is produced by combining or mixing one or more of the chemical compounds provided above with a pharmaceutically acceptable carrier in accordance with methods known to those skilled in the art.

Suitable carriers include artificial and biological delivery systems such as, but not limited to liquids, gels, suspensions, emulsions, dendrimers, quantum dots, hydrogels, aerogels, foams and creams. Suitable carriers also include nano drug delivery carriers such as, but not limited to, nanospheres, hydrogels with and without nanoparticles, nanocapsules, nanotubes, and nanoparticles. Microsystems are also suitable carriers such as, but not limited to, patches and micropumps. Vesicles of biological or inert origin are also included in the list of suitable carriers, such as, but not limited to, liposomes, aquasomes, niosomes, ethosomes, polymersomes and cubosomes. In addition, carriers having a biological origin such that they provide a class of macromolecules including, but are not limited to, lipids, carbohydrate structures, proteins, peptides and nucleic acids.

### Administration of Pharmaceutical Compositions

The pharmaceutical composition may be administered in any suitable manner known to those skilled in the art. Suitable delivery systems include passive delivery systems, such drug delivery via diffusion, or active delivery systems, such as drug delivery via digestion. The pharmaceutical composition may be administered orally, in liquid form or in the form of a solid, such as a pill, capsule or powder; intravenously, topically, subcutaneously, vaginally or rectally, such as with a medicated suppository; via ocular means, such as eye drops, ointments or medicated contact lenses; via transdermal means, such as a patch, via pulmonary means, such as an inhaler, via microelectrochemical systems, or via micropumps. With the potential for nanoparticle carriers, many options for administration exist and are known to those skilled in the art.

Oral formulations of the pharmaceutical composition may be administered to a patient at suitable dose levels calculated to achieve the desired chemotherapeutic effect. In one embodiment, an oral formulation having a 10-1000 μg/mL concentration of the compound of Formula 1 or 2 in an aqueous solution may be dosed daily at between 0.1 ounce per 1000 pounds body weight (approximately 0.065 mL/kg of patient weight) and 1 ounce per 1 pound (approximately 6.5 mL/kg of patient weight). In some embodiments, the oral formulation is administered at a daily dosage of 1 ounce per 100 lb. of patient weight (approximately 0.65 mL/kg). The oral formulation may be provided in a single daily dose or in two or more smaller doses in a day. If the pharmaceutical composition exhibits increased potency, the pharmaceutical dose is reduced accordingly. In another embodiment, the dose of the pharmaceutical composition is a range of from 0.001 mL/100 lbs. body weight to 3 ml/100 lbs. body weight. In another embodiment, the dose of the pharmaceutical composition is 0.03 mL per 100 pounds of body weight.

The pharmaceutical composition may be administered with other therapies. For example, the pharmaceutical composition may be administered to a patient simultaneously or sequentially with a second therapeutic agent, such as, but not limited to, a chemotherapeutic drug or a steroid, antibiotic, vitamin, antibody therapy, gene therapy or the like.

The invention will now be further described by reference to the following non-limiting examples

### EXAMPLES

### Example 1: Chaga Ethanol Extraction

Chaga mushrooms were chopped using a coffee grinder into pieces smaller than about 3 mm. One part chopped Chaga was mixed with two parts ethanol. Water was added to the ethanol/Chaga mixture in a volume ratio of 70:30 ethanol/water. The water/ethanol/Chaga mixture was then left to sit at room temperature for a period of three months, at which time the mixture was filtered using a sieve with an aperture size of around 1 mm so that substantially all solid matter was removed. After filtering, the mixture was stored in a glass jar as the ethanol extraction.

### Example 2: Chaga Reduction

Chaga mushrooms were chopped using a coffee grinder into pieces smaller than about 3 mm. About 1.5 cups of the chopped Chaga was placed in a pot containing around 4.8 liters of room temperature water. The water had been reverse osmosis filtered. The water was heated and brought to a strong, rolling boil for 4-5 minutes and then the temperature reduced to maintain a low boil for about 20-30 minutes. The heat was removed, and the water allowed to cool for about thirty minutes, so that the cycle of heating and cooling took about one hour. The cycle was repeated five to six times over a period of 5-6 hours. The Chaga/water mixture was filtered using a sieve with an aperture size of around 1 mm so that substantially all solid matter was removed from the water. After cooling and filtering the mixture was stored as the Chaga reduction.

### Example 3: Extraction/Reduction Mixture

One part by volume of the ethanol extraction was added to five parts by volume of the Chaga reduction in a glass container to form the extraction/reduction mixture. The extraction/reduction mixture is also referred to below as “the oral formula solution.”

### Example 4: Distillate

The extraction/reduction mixture was distilled by heating the mixture up to a temperature where steam was first detected to come off the mixture. The matter coming off the mixture was cooled and condensed to form the distillate. The amount of distillate achieved was in the range 30-45 ounces, from an ER mixture of 384 oz. Thus, the remaining fraction was present in an amount of 339-354 ounces.

### Example 5: Oral Formulation I

Oral Formulation I was prepared by mixing the reduction, the extract and the remaining fraction in a weight ratio of 5:1:1.

### Example 6: Oral Formulation II

Oral Formulation II was prepared by mixing the reduction, the extract, the remaining fraction, the manuka honey and the coconut oil in a weight ratio of 10:2:2:1:1.

### Example 7: Topical Formulation I

Equal portions by volume of remaining fraction and ethanol extraction were mixed together to form an intermediate mixture to which was added manuka honey in a volume ratio of 8 parts intermediate mixture and one part manuka honey. The mixture with honey was stirred to dissolve the honey. Coconut oil was then added to the honey-containing mixture, about 1 part coconut oil to 1 part honey-containing mixture, by volume, to make Topical Formulation I.

### Example 8: Topical Formulation II

Formulation II is prepared using the same procedure as Formulation I, except that the intermediate mixture contained two parts Chaga reduction to one part distillate and one part ethanol extraction.

### Example 9: Method for Production of the Compound of Formula 1

1. Prepare Chaga extraction as follows:

Create a ratio of 70/30 up to 50/50 H₂O/EtOH Chaga extraction created by mixing 1 gallon ethanol solution and 1 lbs ground chaga (1:4 up to 1:3 parts chaga to water ethanol solution) in a sealed glass vessel and allow extraction to occur for a minimum of 72 hours up to 60 days at ambient temperature. At the end of extraction period particulates are hand filtered out using a fine mesh filter to remove all solid material.

2. Prepare Chaga reduction using ratio of 100 L reverse osmosis filtered water and 7-8 lbs+/−3 lbs. of chopped and ground chaga.

a. This water/chaga mixture is heated to approx. 160 F-190 F and maintained for 3-7 days under slight vacuum in glass reactor vessel at 5-15 PSI+/−4 PSI. This forms the Chaga reduction.

The Chaga reduction will lose up to 5% volume through evaporation/vaporization.

b. Once Chaga reduction has condensed for 3-7 days the chaga reduction is transferred from the glass reactor to a stainless steel still fitted with a copper helmet. (Increased concentration of betulinic acid is achieved).

c. Chaga reduction is then distilled for 3-7 hours and will produce 1-5 gallons of distillate. Distillation occurs at approx. 210° F.+/−10° F. at 20-35 PSI+/−10 PSI (increases functionality of new reaction sites on betulinic acid by altering boiling point of betulinic acid).

3. After distillation, the distillate is set aside and the remaining fraction, referred to herein as the reduction, the portion of the distillation process not converted to distillate, is used for the reaction. The volume of this remaining fraction, or reduction, will be approximately 80% of the starting volume. The temperature of the fraction during the reaction will stay at approximately 190° F.+/−10° F.

a. Once reduction has been removed from the still it is mixed in the following quantities for the final reaction:
4 gallons reduction (also referred to herein as the remaining fraction)
250-400 g Manuka Honey
115-120 mL Medium Chain Triglyceride oil (MCT oil)
5 mL 70/30 H2O/Eth Chaga extraction (from step 1)

b. All ingredients are mixed together in stainless steel vessel where reaction begins. The initial indication of reaction occurring is the giving off of “sparks” of light, the formation of micelles and giving off of heat and gas.

c. The mixture is then bottled to contain and induces the continued reaction for 4-6 hours while it cools slowly to room temperature.

d. The mixture can be stored at ambient temperature for 3-4 weeks without degradation.

The foregoing process resulting in the production of a bioactive Chaga compound having enhanced bioavailability and has the chemical structure of Formula 1 below. This Chaga compound has a structure similar to that of betulinic acid. However, it exhibits high bioavailability in vitro and in vivo. Betulinic acid is only 2% bioavailable in its natural form. The compound having the chemical structure of Formula 1 exhibits 98% bioavailability.

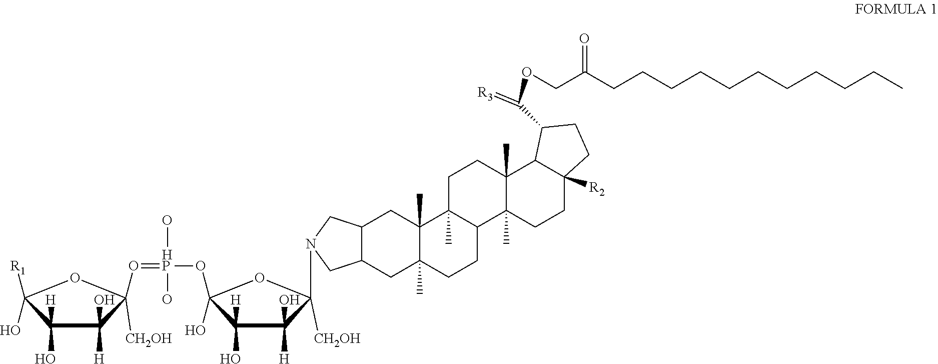

### Example 10: Method for Production of the Compound of Formula 2

1. Prepare Chaga reduction using ratio of 100 L reverse osmosis filtered water and 7-8 lbs+/−3 lbs. of chopped and ground chaga.

a. This water/chaga mixture is heated to approx. 160 F-190° F. and maintained for 3-7 days under a slight vacuum in glass reactor vessel at 5-15 PSI+/−4 PSI. This forms the Chaga reduction.

Note: The Chaga reduction will lose up to 5% volume through evaporation/vaporization.

b. Once the Chaga reduction has been condensed for 3-7 days, the Chaga reduction is transferred from the glass reactor to a stainless steel still fitted with a copper helmet. (Increased concentration of betulinic acid is achieved).

c. Chaga reduction is then distilled for 3-7 hours and will produce 1-5 gallons of distillate. Distillation occurs at approximately 210° F.+/−10° F. at 20-35 PSI+/−10 PSI (this increases functionality of new reaction sites on betulinic acid by altering the boiling point of betulinic acid).

2. After distillation, the distillate is set aside and the remaining fraction, referred to herein as the reduction, the portion of the distillation process not converted to distillate, is used for the reaction. The volume of this remaining fraction, or reduction, will be approximately 80% of the starting volume. The temperature of the fraction during the reaction will stay at approximately 190 F+/−10° F.

Note: Chaga reduction to fraction as prepared in Formula 1 is used as starting material.

3. In a 300 mL Parr reaction vessel at room temperature mix:
200 mL of the Chaga fraction betulinic acid concentration or other Chaga constituents capable of reacting with the following:
15 g Proline or other amino acids
15 g Fructose or other reducing sugar
5 g Sunflower lecithin (or other surfactant and/or fatty acid)

4. Stir gently. Starting material will begin to crystalize at room temperature.

5. Seal reaction vessel according to manufacturer guidelines.

6. Heat mixture in fully sealed reaction chamber at 405° F.+/−15° F. at 35 PSI+/−10 PSI.

Note: Steps 3-6 will take approximately 30-50 minutes.

7. Once temperature/pressure has been achieved remove the heat source and allow vessel to cool to a max of 90 F before degassing.

8. Once at correct temperature, open pressure release valve and slowly de-gas chamber.

9. Open vessel and separate liquid from solid crystal phases. Liquid phase contains concentration of the fully synthesized compound of Formula 2.

The foregoing process results in the production of a bioactive Chaga compound having enhanced bioavailability and has the chemical structure of Formula 2 below. This Chaga compound has a structure similar to that of betulinic acid. However, it exhibits much higher bioavailability characteristics.

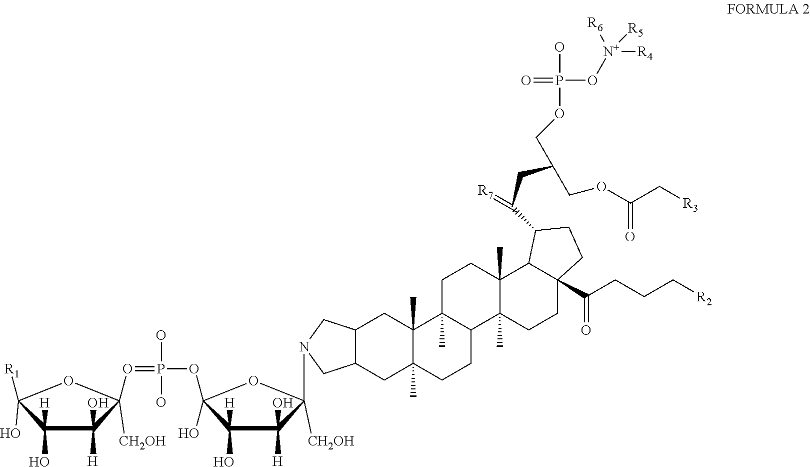

### Example 11: Mitochondria Study

More than just the powerhouse of the cell, mitochondria are not only a hub of energy transformation but may very well prove to be the conductors of an intricate chorus of translation between several energetic forms that are responsible for all of biological life. These tiny organs of facilitated reactions may actually have the capacity to determine the fate of the entire organism. They may in fact be the timekeepers for the cell, and through the cell the entire organism.

Should this be the case, these functions would need to be tightly controlled, as errors or malfunctions could lead to short-term disaster or long-term disease. But what if one had a way to not only support these functions, but to create a larger range of safe operation, allowing for these tiny powerhouses to increase the life and health span of any organism to which they belong. This could lead to disease reversal and long-term increases to both health and lifespan.

Mitochondria have the capacity to both direct the fate of stein cells required for organismal life as well as hold the keys for initiating the apoptotic cycle at the end of the cell's lifespan. Research has shown that mitochondria are not only suppliers of energy but hold a critical role in cellular signaling and cell fate determination. The hypothesis that mitochondria could be communicating not only with mammalian cells but also microorganisms that share a symbiotic relationship with their host is also coming to light with many research efforts.

Mytulin Restore™ (commercially available from Mytosynth Nutraceuticals, Bloomington, MN), a dietary supplement containing an active ingredient that is derived from a Chaga mushroom preparation as described above in Example 9, was originally designed with the intent to support mitochondrial health through direct and indirect means. By supporting the microbiome in ways that support healthy cellular function and providing substrates immediately beneficial to the mitochondria of all cells that contain them, Mytulin Restore offers a unique way to approach whole-body health and longevity.

In the study the original hypothesis that Mytulin Restore affects mitochondrial function by increasing the Mitochondrial Membrane Potential (MMP) capacity, supporting the mitochondria for more efficient ATP synthesis within a safe and optimal range for the least amount of stress response from the mitochondria is explored. This initiates a cascade of events, both to increase the health span of an organism on a cellular level and to support healthy apoptosis in cells that are past their correct lifespan.

### Experiment:

Fifteen volunteers were secured. All were healthy adults. Signed written consent forms were obtained from all 15 volunteers. The protocol consisted of a 7-day treatment using 4 oz of Mytulin Restore per day for 7 days with no additional control criteria other than exclusionary criteria outlined in the study protocol. Baseline and 7-day blood samples were obtained, and lymphocytes were isolated and purified for measurement by Bio Assay Systems (Hayward, CA). Changes to MMP measurements were obtained to ascertain if Mytulin Restore had any effect on the MMP of healthy lymphocytes in humans.

### Detailed Protocol:

1) Fifteen whole blood samples with heparin were received one day after having been collected and shipped overnight at 4° C.

2) The samples were transferred in two batches (sequentially, 8 then 7) to 15 mL Falcon tubes then processed following the STEMCELL Tech's EasyEight's protocol for 3.5 mL, volume samples. Each sample received 175 μL of the Isolation cocktail and 175 μL of RapidSpheres™ beads. All samples were brought to 5 mL with D-PBS (STEMCELL Tech, Vancouver, BC—Cat #37350). The final purification steps were done in 5 mL Falcon tubes.

3) Cells were then counted manually to obtain a cells/μL count.

4) For the Mitochondrial Membrane Potential experiment, 300K cells from each subject were resuspended in a final volume of 600 μL using a KCl solution (120 mM KCl, 3 mM HEPES, 5 mM KH2PO4, 3 mM MgSO4, 1 mM EGTA at pH 7.2).

5) The cells were treated with Digitonin at a final concentration of 0.1 mg/mL then subdivided and treated with either 72.5 μL of KCl solution or 72.5 μL of 5 μM Carbonyl cyanide 3-chlorophenylhydrazone (CCCP) in KCl solution.

6) Following treatment with Digitonin and CCCP, 100 μL of each treatment was transferred to a black flat, clear-bottom 96-well tissue culture-treated fluorescence plate, sealed, and incubated at 37° C. for 30 minutes.

7) Following the 30′ incubation, each well received 10 μL of KCl solution with 1.65 μM Tetramethylrhodamine, Methyl Ester, Perchlorate (TMRM) and 50 mM Succinate and was incubated for a further 5 minutes at RT.

8) The plates were centrifuged at 800×g for 2 minutes and the solution removed. The cells were then washed with 100 μl of 1×PBS pH 7.2. The wash was repeated three (3) more times with the final cells resuspended in 100 μL of PBS.

9) The plate was imaged at 530 nm/580 nm (550 cutoff) for 10 minutes in kinetic mode.

10) Analysis was completed in Excel. Each sample time point was averaged then the time points were rolled up into an average from eleven (11) reads. The difference and ratio were calculated. In addition, the averaged values for each time point and treatment were treated as an array and subjected to T. Test (one tailed, homoscadastic) using a p-value <0.5.

### Summary

An exploratory pilot experiment was completed to examine mitochondrial membrane potential using a modified approach that was based on methods described in Huang F (2002) Development of a High Throughput Screening Assay for Mitochondrial Membrane Potential in Living Cells. Journal of Biomolecular Screening. 7:383-389, and Pecina et al. (2014). Noninvasive diagnostics of mitochondrial disorders in isolated lymphocytes with high resolution respirometry. BBA Clinical 2:62-71, both of which are incorporated by reference herein. In brief, total human lymphocytes were purified from heparin-treated whole blood using STEMCELL Technologies' EasySep™ System (Cat #19655) with the EasyEights™ magnet. The blood samples were obtained from 15 volunteers from a clinical site on two different days, seven (7) days apart. BioAssay Systems was blind to the treatment and collection of the blood samples.

The Mitochondrial Membrane Potential was measured in 96-well plates following treatment with Tetramethylrhodamine, Methyl Ester, Perchlorate (TMRM) in the presence or absence of Carbonyl cyanide 3-chlorophenylhydrazone (CCCP). The TMRM is retained in the mitochondria if there was membrane potential. CCCP releases the membrane potential, so the differential provides some indication of the level of mitochondrial membrane potential. The fluorescence results were time-averaged then examined for differential by difference (−CCCP RFU−+CCCP RFU), by normalization (−CCCP RFU/+CCCP RFU), and using the T-Test (one-tailed, homoscedastic analysis of the averaged RFUs over time, p-value <0.05).

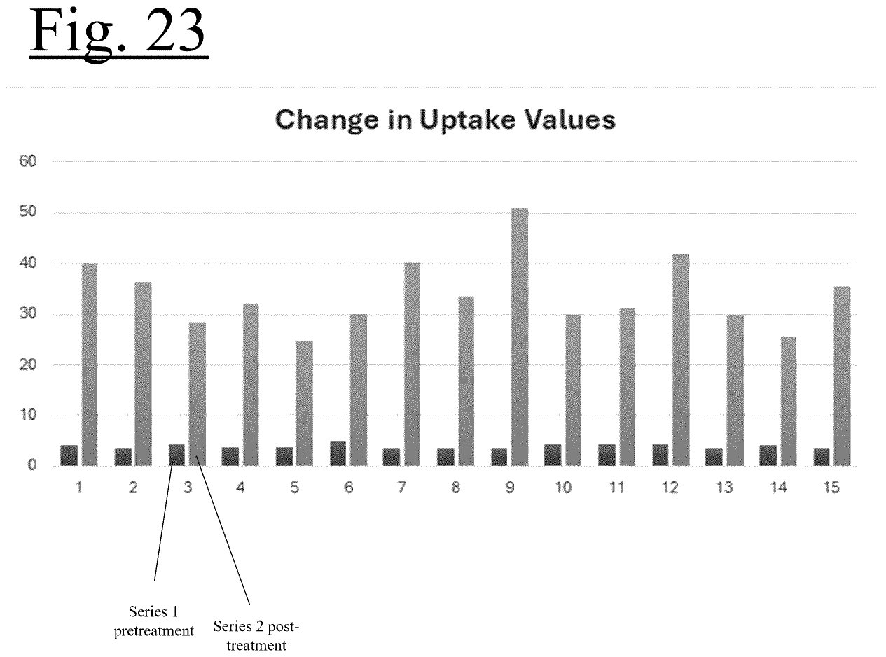

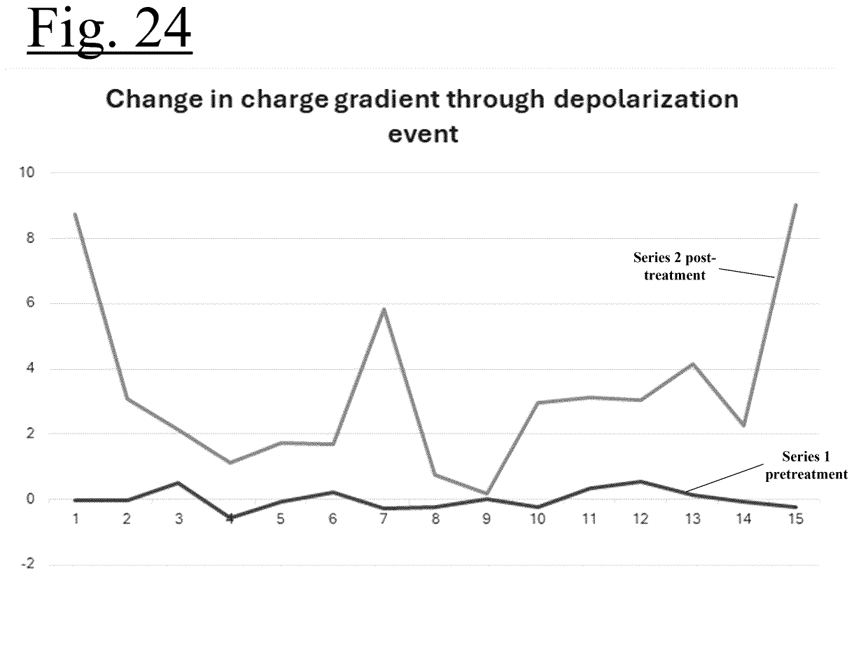

The results are shown in FIGS. 23 and 24.

FIG. 23 shows that Mytulin Restore increases the mitochondrial capacitance—a change (increase) in uptake values of TMRM).

FIG. 24 shows the Mytulin Restore increases the efficiency of the mitochondrial membrane to maintain the charge gradient. An increased change in differential in depolarized mitochondrial membrane. Mytulin Restore reduces ROS signaling/Proton leak.

### CONCLUSION

If Mytulin Restore increases the capacitance of the mitochondria, and ATP synthesis increases proportionally with the improved efficiency and does not increase H+ leak or ROS outputs, then the safe range of oscillation between ATP synthesis and ATPase/MMP maintenance must also increase This would explain the significant increase in uptake values and differential change measured before and after depolarization of the mitochondrial membrane. These findings provide a glimpse into the more intricate functions of the compounds found in Mytulin Restore. The components of Mytulin Restore may be increasing the capacitance of the mitochondria by providing substrates that, upon entering the mitochondria, are able to increase the dielectric constant in the inner membrane enough to provide for a safe increase in the range of the charge gradient maintained by the mitochondria by allowing the mitochondria to hold more charge. This, in turn, increases the range (distance travelled) in the microenvironment of each system where the mitochondria were able to provide enough resources (sufficient amounts of ATP) to better stabilize the phosphate buffer system and protect the cell for optimal functioning.

As noted above, the present invention is applicable to chemical compounds and pharmaceutical compositions containing the chemical compounds described herein and methods of making and using thereof. Accordingly, the present invention should not be considered limited to the examples described above but rather should be understood to cover all aspects of the invention as fairly set out in the attached claims.

Various modifications, improvements and equivalents will be readily apparent to those of skill in the art to which the present invention is directed upon review of the present specification. The claims are intended to cover such modifications, improvements and equivalents. Those skilled in the art will recognize or be able to ascertain using no more than routine experimentation, many equivalents to the specific embodiments of the invention described herein. Such equivalents are intended to be encompassed by the following claims.

What is claimed is:

1. A method of increasing the bioavailability of a triterpene, comprising:
a. combining a reducing sugar with an amino acid,
b. heating the combination at an elevated temperature under vacuum for a sufficient amount of time to form one or more Maillard reaction products,
c. adding the one or more Maillard reaction products to a triterpene to produce a triterpene reaction mixture,
d. boiling the reaction mixture under vacuum, and
e. esterifying the reaction mixture with a fatty acid under to produce an emulsion, wherein the emulsion comprises a modified triterpene having increased bioavailability.

2. A bioavailable triterpene produced by the method of claim 1.

3. A bioavailable triterpene having the chemical formula of Formula 1 or Formula 2.

4. A bioavailable triterpene, wherein the bioavailable triterpene is synthetically produced.

5. A bioavailable triterpene in combination with a therapeutic drug.

6. The combination of claim 5, wherein the therapeutic drug is a chemotherapeutic agent, analgesic, neurotransmitter, opioid, hormones or corticosteroid.

7. A pharmaceutical composition comprising the bioavailable triterpene of claims 2-6 in a pharmaceutically-acceptable carrier.

8. A method of treating a disease, comprising administering the pharmaceutical composition of claim 7 to a human or animal having the disease.

9. The method of claim 8, wherein the disease is cancer.

10. A method for increasing the bioavailability of a therapeutic drug by combining the therapeutic drug with a bioavailable triterpene.

11. The method of claim 10, wherein the bioavailable triterpene is the bioavailable triterpene of claims 2-4.

12. The method of claim 10 wherein the therapeutic drug is a chemotherapeutic agent, analgesic, neurotransmitter, opioid, hormones or corticosteroid.

13. A pharmaceutical composition comprising a combination of a bioavailable triterpene and a therapeutic drug in a pharmaceutically-acceptable carrier.

14. The pharmaceutical composition of claim 13, wherein the bioavailable triterpene is produced by the method of claim 1.

15. The pharmaceutical composition of claim 13, wherein the bioavailable triterpene has the chemical formula of Formula 1 or Formula 2.

16. The pharmaceutical composition of claim 13, wherein the bioavailable triterpene is synthetically produced.

17. The pharmaceutical composition of claim 13 wherein the combination is a mixture of a bioavailable triterpene and a therapeutic drug in a pharmaceutically-acceptable carrier.

18. The pharmaceutical composition of claim 17, wherein the bioavailable triterpene is produced by the method of claim 1.

19. The pharmaceutical composition of claim 17, wherein the bioavailable triterpene has the chemical formula of Formula 1 or Formula 2.

20. The pharmaceutical composition of claim 17, wherein the bioavailable triterpene is synthetically produced.

21. The pharmaceutical composition of claim 13 wherein the combination is a complex of a bioavailable triterpene and a therapeutic drug in a pharmaceutically-acceptable carrier.

22. The pharmaceutical composition of claim 21, wherein the bioavailable triterpene chemical compound is complexed to the therapeutic drug with a saccharide or polysaccharide.

23. The pharmaceutical composition of claims 21-22, wherein the bioavailable triterpene is produced by the method of claim 1.

24. The pharmaceutical composition of claims 21-22, wherein the bioavailable triterpene has the chemical formula of Formula 1 or Formula 2.

25. The pharmaceutical composition of claims 21-22, wherein the bioavailable triterpene is synthetically produced.

26. The pharmaceutical composition of claims 13-25 wherein the therapeutic drug is a chemotherapeutic agent, analgesic, neurotransmitter, opioid, hormones or corticosteroid.

27. A method of treating a disease, comprising administering the pharmaceutical composition of claims 13-26 to a human or animal having the disease.

28. The method of claim 27, wherein the disease is cancer.

29. A method of increasing the bioavailability of a bioactive Chaga mushroom isolate comprising:
forming a reduction of Chaga mushroom in a reducing solvent;
forming an extraction of Chaga mushroom in an extraction solvent;
mixing the reduction of Chaga mushroom with the extraction of Chaga mushroom, and isolating a bioactive chemical compound, wherein the compound has increased bioavailability when compared with a naturally-occurring bioactive Chaga mushroom isolate.

30. A bioactive chemical isolate of a Chaga mushroom having enhanced bioavailability prepared by a method comprising forming a mixture of a Chaga mushroom reduction and a Chaga mushroom extraction with an esterification mixture comprising proline, fructose and a fatty acid, and an emulsifying sugar ester capable of emulsion within otherwise non-miscible liquids to facilitate emulsion at a temperature that results in a reaction between the Chaga mushroom reduction and Chaga mushroom extraction mixture with the esterification mixture to produce a reaction mixture, and combining the reaction mixture with a medium chain triglyceride oil to make the Chaga mushroom isolate having enhanced bioavailability.

31. The method of claim 29, wherein the esterification mixture is a Manuka honey.

32. A method of making a Chaga mushroom isolate having enhanced bioavailability comprising:
preparing a Chaga mushroom reduction in a reduction solvent under high pressure;
preparing a Chaga mushroom extraction in an extraction solvent;
mixing the Chaga reduction with the Chaga extraction;
reacting the Chaga reduction and Chaga extraction mixture with an esterification mixture comprising proline, fructose and a fatty acid, and an emulsifying sugar ester capable of emulsion within otherwise non-miscible liquids to facilitate emulsion at a temperature that results in a reaction between the Chaga mushroom reduction and Chaga mushroom extraction mixture with the esterification mixture to produce a reaction mixture and combining the reaction mixture with medium chain triglyceride oil to make the Chaga mushroom isolate having enhanced bioavailability when compared with a naturally-occurring bioactive Chaga mushroom isolate.

33. The method of claim 32, wherein the esterification mixture is a Manuka honey.
